(19) United States
(12) Patent Application Publication
(10) Pub. No.: US 2004/0209928 A1
(43) Pub. Date: Oct. 21, 2004

(54) Glucagon receptor antagonists/inverse agonists

(76) Inventors: Ravi Kurukulasuriya, Gurnee, IL (US); James T. Link, Evanston, IL (US); Jyoti R. Patel, Libertyville, IL (US); Bryan K. Sorensen, Antioch, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/743,954

(22) Filed: Dec. 23, 2003

Related U.S. Application Data
(60) Provisional application No. 60/437,132, filed on Dec. 30, 2002.

(51) Int. Cl. A61K 31/427; A61K 31/422; A61K 31/4178; C07D 417/02; C 07D 4 3/02

(52) U.S. Cl. 514/365; 514/374; 514/389; 514/381; 548/146; 548/215; 548/253; 548/311.1

(57) ABSTRACT

Compounds of formula (I)

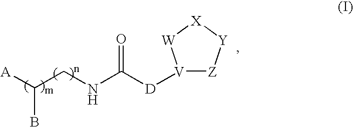

or pharmaceutically suitable salts thereof are novel glucagon receptor antagonists or inverse agonists and are useful for treating type 2 diabetes in a mammal.

This application claims priority to the provisional application Serial No. 60/437,132 filed on Dec. 30, 2002.

### FIELD OF THE INVENTION

The present invention relates to novel compounds which antagonize the action of the glucagon hormone on the glucagon receptor. In particular, the present invention is directed to glucagon antagonists or inverse agonists and methods for using such compounds to treat mammals which would benefit from glucagon receptor antagonism.

### BACKGROUND OF THE INVENTION

Type 2 diabetes (also referred to as non insulin-dependent Diabetes Mellitus) is a debilitating disease characterized by an abnormal elevation of blood glucose levels driven by three factors: increased hepatic glucose production, inadequate clearance of glucose via insulin mediated pathways, and decreased uptake of circulating glucose by tissues (DeFronzo, Diabetes Review 5(3), 177-269, (1997)). Administration of agents that decrease hepatic glucose production are a fundamental approach to controlling blood glucose (De Feo et al., Am. J. Physiol. 257, E35-E42 (1989); Rooney, et al., J. Clin. Endocrinol. Metab. 77, 1180-1183 (1994); and Dinneen et al., J. Clin. Invest., 92, 2283-2290 (1993)).

Glucagon has been shown to have a major influence on glucose production. Glucagon is a 29-amino acid peptide that is the primary counter-regulatory hormone to insulin. Secreted by alpha cells in the pancreas, glucagon binds to a G-protein coupled receptor residing primarily in liver. Binding to the glucagon receptor activates the Gₛ family of guanine nucleotide binding proteins, stimulates adenylate cyclase, and increases cyclic AMP formation. This second messenger in turn activates a cAMP-dependent protein kinase cascade that stimulates hepatic glucose output by enhancing glycogenolysis and gluconeogenesis, while inhibiting glycogen synthesis and glycolysis (Unger and Orci, Joslin's Diabetes Mellitus, Kahn and Weir, Eds. Baltimore, Williams and Wilkins, 163-176 (1994)). In type 2 diabetics, fasting hyperglycemia is closely associated with increased rates of glucose production which can be ascribed to increased rates of gluconeogenesis. In addition, defects in early insulin secretion in response to a meal lead to increased levels of glucagon, resulting in elevated hepatic glucose output and plasma glucose. A glucagon receptor antagonist would be expected to reduce hepatic glucose output and thus aid in glycemic control.

Glucose homeostasis is maintained through a balance between insulin and glucagon. There is evidence to indicate that subjects with type 2 diabetes are hyperglycemic due to a relative deficiency in the insulin to glucagon ratio (Unger, Metabolism, 27, 1691 (1978); Unger, Diabetologia, 28, 574-578 (1985); and Unger and Orci, Lancet, 1, 14-16 (1975)). Studies have shown that type 2 diabetic patients are hyperglucagonemic, have greater rates of gluconeogenesis than normal subjects (Dobbs, Science, 187 544-547 (1975); Magnusson J. Clin. Invest. 90, 1323 (1992); Reaven, J. of Clin. Encocrin. And Metab. 64, 106-110 (1987); Rizza, J. Clin. Invest. 63, 1119-1123 (1979); and Baron, Diabetes, 36, 274-283 (1987)) and exhibit delayed early (<30 min) insulin secretion in response to glucose challenge or meal consumption (Shah, J. Clin. Endocrinol. Metab, 85, 4053 (2000)). The delayed insulin response results in hyperglycemia by impairing glucose- and insulin-induced inhibition of glucagon secretion. Lack of suppression of serum glucagon levels increases gluconeogenesis, glycogen breakdown and increases hepatic glucose output (Shah, Am. J. Physiol. 277, E283-E290 (1999)). Unlike subjects with type 2 diabetes, high glucagon levels do not lead to hyperglycemia in non-diabetics whose early insulin response is normal. Therefore, higher glucagon levels that result from lack of an early insulin response are an important factor in dysregulation of glycemic control. Defective suppression of glucagon release by early insulin release is also a feature of subjects who are prediabetic but show impaired glucose tolerance (Ahren and Larsson, Diabetologia, 37, 985-993 (1994)). These findings are consistent with the notion that a relative excess of glucagon to insulin contributes to hyperglycemia in type 2 diabetes, and agents that block glucagon action would be beneficial in the treatment of type 2 diabetes.

Several lines of experimental evidence suggest that therapeutic interventions focusing on suppression of glucagon action will result in lowering of hyperglycemia. Monoclonal antibodies that deplete plasma glucagon have successfully moderated hyperglycemia in streptozotocin (STZ)-treated rats (Brand, Diabetologia, 37, 985-993 (1994)), alloxan diabetic rabbits (Brand, Diabetes, 45, 1076-1083 (1996)), and, recently, in ob/ob mice (Brand, American Diabetes Association poster session in San Antonio, Tex., USA (2000)). Sub-chronic antibody treatment of ob/ob mice lowered plasma glucose from 12.3 mM to 9.1 mM and resulted in a decrease of HbA_{1c} from 8.1 to 7.1%. A peptidic glucagon antagonist, ALT 3000, decreased initial fasting glucose in STZ-diabetic rats by 60% (Terleckyj, Diabetes, 45, 220A (1996)). The evidence that glucagon receptor antagonists can regulate glycemic levels has been extended to humans through a phase I clinical study with BAY-279955 (Peterson and Sullivan, Diabetologia, 44, 2018 (2001)). In this study normoglycemic subjects were subjected to a somatostatin “clamp” and challenged with a continuous infusion of exogenous glucagon, leading to an increase in hepatic glucose output and plasma glucose. Subjects that received the antagonist showed no increase in hepatic glucose output and a markedly blunted rise in plasma glucose concentration. Taken together, these studies suggest that suppression of glucagon activity may represent an effective therapy for diabetes.

The glucagon antagonist can be peptidic or non-peptidic. Peptide antagonists of peptide hormones are often quite potent. However, they are generally known not to be orally available because of degradation by physiological enzymes, and because of poor distribution in vivo. Therefore, orally available non-peptide antagonists of peptide hormones are generally preferred.

Several publications disclose peptides that are stated to act as glucagon antagonists. One of the most thoroughly characterized antagonists is DesHis¹[Glu⁹]-glucagon amide (Unson, Peptides 10, 1171 (1989); and Post, Proc. Natl. Acad. Sci. USA, 90, 1662 (1993)). Other antagonists include DesHis¹, Phe⁶[Glu⁹]-glucagon amide (Azizh, Bioorganic & Medicinal Chem. Lett., 16, 1849 (1995)) and Nleu⁹,Ala^{11,16}-glucagon amide (Unson, J. Biol. Chem., 269, 12548 (1994)). A monoclonal antibody which is a competitive antagonist of the glucagon receptor has also been described (Bywater, D56, 573-580 (2000)).

Glucagon receptor antagonists/inverse agonists may be useful for treating mammals suffering from type 2 diabetes and for treating symptoms of type 2 diabetes including hyperglycemia, inadequate glucose clearance, obesity, hyperinsulinemia, and hypertriglyceridemia. In addition to type 2 diabetes, glucagon receptor antagonists/inverse agonists may be useful to treat diseases such as type 1 diabetes, obesity, and Syndrome X.

Several publications disclose nonpeptides that are stated to act as glucagon antagonists/inverse agonists including: Madsen, J. Med. Chem., 41, 5150-5157 (1998); Parker, Diabetes, 49, 2079-2086 (2000); Ling, J. Med. Chem., 44, 3141-3149 (2001); Ling, Bioorganic & Medicinal Chem. Lett. 12, 663-666 (2002); Rault, Eur. J. Med. Chem., 293-308 (1998); Cascieri, J. of Biol. Chem. 1999, 274, 8694-8697 (1999); Laszlo, Bioorganic & Medicinal Chem. Lett., 9, 641-646 (1999); Bioorganic & Medicinal Chem. Lett., 11, 2549-2553 (2001)); Ladouceur, Bioorganic & Medicinal Chem. Lett., 12, 461-464 (2002); Smith, Bioorganic & Medicinal Chem. Lett., 12, 1303-1306 (2002); and Ladouceur, Tetrahedron Lett., 43, 4455-4458 (2002).

The following references discloses glucagon receptor antagonists/inverse agonists for the treatment and/or prevention of glucagon-mediated conditions and diseases such as hyperglycemia, type 1 diabetes, type 2 diabetes, lipid metabolism disorders and obesity: WO 00/69810; WO 02/00612; WO 02/40444 A1; WO 02/40445 A1; WO 02/40446 A1; WO 99/01423; WO 00/39088.

The following references disclose glucagon receptor antagonists and inhibitors of the biosynthesis and action of TNF-α and IL1 and may be used as antidiabetic agents as well as treatments for other cytokine mediated diseases: U.S. Pat. No. 5,776,954; WO 97/16442; WO 98/22108 The following references disclose glucagon receptor antagonists for the treatment of diabetes, obesity, hypertension, cachexia, and other disease states mediated by elevated glucagon levels: WO 98/21957; WO 98/22109; U.S. Pat. No. 5,880,139; U.S. Pat. No. 6,218,431 B1.

Although there are glucagon antagonist/inverse agonist therapies in existence, there continues to be a need for improved glucagon antagonist/inverse agonist. Thus, the identification of compounds which are glucagon receptor antagonists/inverse agonists such as compounds of the present invention provide an alternative method of treatment for those suffering from conditions such as hyperglycemia, type 1 diabetes, type 2 diabetes, lipid metabolism disorders, syndrome X and obesity.

### SUMMARY OF THE INVENTION

In its principle embodiment, the present invention discloses compounds of formula (I) that are useful as glucagon antagonists or inverse agonists:

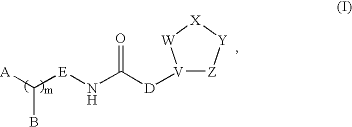

or a pharmaceutically suitable salt, ester or prodrug thereof, wherein

A is selected from the group consisting of CO₂H and tetrazole;

B is selected from the group consisting of H, F, OH, alkoxy and NRₐR_{b} wherein Rₐ and R_{b} are each independently selected from the group consisting of hydrogen, alkyl, alkylcarbonyl, alkylsulfonyl alkoxyalkyl, cycloalkyl, cycloalkylcarbonyl, cycloalkylsulfonyl, cycloalkylalkyl, heterocycle, heterocyclealkyl, heterocyclecarbonyl and heterocyclesulfonyl;

D is selected from the group consisting of aryl and heteroaryl;

E is (CH₂)ₙ;

m and n are each independently 0, 1, or 2;

V is selected from the group consisting of —C(R_{c})— and —N—, wherein R_{c} is selected from the group consisting of hydrogen, alkyl, alkoxy, alkoxyalkyl, cycloalkyl, cycloalkyloxy, cycloalkylalkyl, heterocycle and heterocyclealkyl;

W is selected from the group consisting of —C(R_{d}Rₑ)—, —(R_{d})N—, —O—, —S—, —S(O)— and —S(O)₂—;

X is selected from the group consisting of —C(O)—, —C(O)C(R_{f}R_{g})—, —C(R_{f}R_{g})C(O)—, —C(S)—, —C(R_{f}R_{g})—, —C(R_{f}R_{g})C(RᵢRⱼ)—, —C═N(Rⱼ)—, —S(O)— and —S(O)₂—;

Y is selected from the group consisting of —C(RₖRₘ)—, —(Rₖ)N—, —O—, —S—, —S(O)— and —S(O)₂—;

Z is selected from the group consisting of a bond, —C(RₚR_{q})— and —C(RₚR_{q})C(RₛRₜ)—; and

R_{d}, Rₑ, R_{f}, R_{g}, Rᵢ, Rⱼ, Rₖ, Rₘ, Rₚ, R_{q}, Rₛ and Rₜ are each independently selected from the group consisting of hydrogen, alkyl, alkoxy, alkoxyalkyl, aryl, arylalkyl, aryloxy, arylalkoxy, cycloalkyl, cycloalkylalkyl, cycloalkyloxy, cycloalkylalkyl, heterocycle, heterocyclealkyl, heterocycleoxy and heterocyclealkoxy.

An additional embodiment of the present invention relates to pharmaceutical compositions comprising a therapeutically effective amount of the compound of formula (I) in combination with a pharmaceutically suitable carrier.

A further embodiment of the present invention relates to antagonizing the effects of glucagon at the glucagon receptor in a mammal comprising administeration of a therapeutically effective amount of the compound of formula (I).

Another embodiment of the present invention relates to a method of treating type 2 diabetes in a mammal comprising administering a therapeutically effective amount of the compound of formula (I).

Another embodiment of the present invention relates to a method of treating symptoms of type 1 or type 2 diabetes including, but not limited to, hyperglycemia, hyperinsulinemia, inadequate, glucose clearance, obesity, hyperlipidemia, lipid metabolism disorders, hypertension and high glucocorticoid levels in a mammal comprising administering a therapeutically effective amount of the compound of formula (I).

### DETAILED DESCRIPTION OF THE INVENTION

### Definition of Terms

As used throughout this specification and the appended claims, the following terms have the following meanings.

The term “agonist,” as used herein, refers to a chemical entity, whether a synthesized chemical entity or a natural endogenous chemical entity, that interacts with the glucagon receptor and elicits an observable biochemical response.

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenyloxy,” as used herein, refers to an alkenyl group, as defined herein, appended to the parent molecular moiety through an oxygen atom. Representative examples of alkenyloxy include, but are not limited to, allyloxy, 2-butenyloxy, 3-butenyloxy and 3-pentenyloxy.

The term “alkenylthio,” as used herein, refers to an alkenyl group, as defined herein, appended to the parent molecular moiety through a sulfur atom. Representative examples of alkenylthio include, but are not limited, allylsulfanyl, 2-butenylsulfanyl, 3-butenylsulfanyl and 3-pentenylsulfanyl.

The term “alkenylene,” denotes a divalent group derived from a straight or branched chain hydrocarbon of from 2 to 10 carbon atoms containing at least one double bond. Representative examples of alkenylene include, but are not limited to, —CH is CH—, —CH is CH₂CH₂—, —CH₂—CH is CH—, —CH₂CH₂CH is CH— and —CH is C(CH₃)CH₂—.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkenyl,” as used herein, refers to an alkoxy group, as defined herein, appended to the parent molecular moiety through an alkenyl group, as defined herein. Representative examples of alkoxyalkenyl include, but are not limited to, 3-methoxy-1-propenyl and 4-methoxy-1-butenyl.

The term “alkoxyalkoxy,” as used herein, refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkoxy,” as used herein, refers to an alkoxyalkoxy group, as defined herein, appended to the parent molecular moiety through another alkoxy group, as defined herein. Representative examples of alkoxyalkoxyalkoxy include, but are not limited to, (2-methoxyethoxy)methoxy and (2-methoxyethoxy)ethoxy.

The term “alkoxyalkoxyalkyl,” as used herein, refers to an alkoxyalkoxy group, as defined herein, appended to the parent molecular moiety through an alkyl group, as defined herein. Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxyalkynyl,” as used herein, refers to an alkoxy group, as defined herein, appended to the parent molecular moiety through an alkynyl group, as defined herein. Representative examples of alkoxyalkynyl include, but are not limited to, 3-ethoxy-1-propynyl and 4-methoxy-1-butynyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkenyl,” as used herein, refers to an alkoxycarbonyl group, as defined herein, appended to the parent molecular moiety through an alkenyl group, as defined herein. Representative examples of alkoxycarbonylalkenyl include, but are not limited to, 3-methoxy-3-oxo-1-propenyl and 3-ethoxy-3-oxo-1-propenyl.

The term “alkoxycarbonylalkoxy,” as used herein, refers to an alkoxycarbonyl group, as defined herein, appended to the parent molecular moiety through an alkoxy group, as defined herein. Representative examples of alkoxycarbonylalkoxy include, but are not limited to, 3-methoxy-3-oxopropoxy, 3-ethoxy-3-oxopropoxy, 2-methoxy-2-oxoethoxy and 4-methoxy-4-oxobutoxy.

The term “alkoxycarbonylalkyl,” as used herein, refers to an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxycarbonylalkynyl,” as used herein, refers to an alkoxycarbonyl group, as defined herein, appended to the parent molecular moiety through an alkenyl group, as defined herein. Representative examples of alkoxycarbonylalkynyl include, but are not limited to, 3-methoxy-3-oxo-1-propynyl and 3-ethoxy-3-oxo-1-propynyl.

The term “alkoxysulfonyl,” as used herein, refers to an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkenyl,” as used herein, refers to an alkylcarbonyl group, as defined herein, appended to the parent molecular moiety through an alkenyl group, as defined herein. Representative examples of alkylcarbonylalkenyl include, but are not limited to, 3-oxo-1-butenyl and 3-oxo-1-pentenyl.

The term “alkylcarbonylalkoxy,” as used herein, refers to an alkylcarbonyl group, as defined herein, appended to the parent molecular moiety through an alkoxy group, as defined herein. Representative examples of alkylcarbonylalkoxy include, but are not limited to, 3-oxopentyloxy, 3-oxobutoxy and 2-oxopropoxy.

The term “alkylcarbonylalkyl,” as used herein, refers to an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonylalkynyl,” as used herein, refers to an alkylcarbonyl group, as defined herein, appended to the parent molecular moiety through an alkynyl group, as defined herein. Representative examples of alkylcarbonylalkynyl include, but are not limited to, 3-oxo-1-pentynyl and 3-oxo-1-pentenyl.

The term “alkylcarbonylalkylthio,” as used herein, refers to an alkylcarbonylalkyl group, as defined herein, appended to the parent molecular moiety through a sulfur atom, as defined herein. Representative examples of alkylcarbonylalkylthio include, but are not limited to, (2-oxopropyl)sulfanyl, (3-oxobutyl)sulfanyl and (3-oxopentyl)sulfanyl.

The term “alkylcarbonyloxy,” as used herein, refers to an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylcarbonylthio,” as used herein, refers to an alkylcarbonyl group, as defined herein, appended to the parent molecular moiety through a sulfur atom. Representative examples of alkylcarbonylthio include, but are not limited to, acetylsulfanyl, propionylsulfanyl and (2,2-dimethylpropanoyl)sulfanyl.

The term “alkylene,” denotes a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkylsulfinyl,” as used herein, refers to an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl,” as used herein, refers to an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl,” as used herein, refers to an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “alkylthio,” as used herein, refers to an alkyl group, as defined herein, appended to the parent molecular moiety through a sulfur atom. Representative examples of alkylthio include, but are not limited, methylsulfanyl, ethylsulfanyl, tert-butylsulfanyl, and hexylsulfanyl.

The term “alkylthioalkoxy,” as used herein, refers to an alkylthio group, as defined herein, appended to the parent molecular moiety through an alkoxy group, as defined herein. Representative examples of alkylthioalkoxy include, but are not limited, 2-methylsulfanylethoxy and 2-ethylsulfanylethoxy.

The term “alkylthioalkyl,” as used herein, refers to an alkylthio group, as defined herein, appended to the parent molecular moiety through an alkyl group, as defined herein. Representative examples of alkylthioalkyl include, but are not limited, methylsulfanylmethyl and 2-(ethylsulfanyl)ethyl.

The term “alkynyl,” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkynyloxy,” as used herein, refers to an alkynyl group, as defined herein, appended to the parent molecular moiety through an oxygen atom. Representative examples of alkynyloxy include, but are not limited to, 3-pentynyloxy, 3-butynyloxy and 2-propynyloxy.

The term “alkynylthio,” as used herein, refers to an alkynyl group, as defined herein, appended to the parent molecular moiety through a sulfur atom. Representative examples of alkynylthio include, but are not limited, 2-propynylsulfanyl, 3-butynylsulfanyl and 3-pentynylsulfanyl.

The term “alkynylene,” denotes a divalent group derived from a straight or branched chain hydrocarbon of from 2 to 10 carbon atoms containing at least one triple bond. Representative examples of alkynylene include, but are not limited to, —C≡C—, —CH₂C≡C—, —CH(CH₃)CH₂C≡C—, —C≡CCH₂—, and —C≡CCH(CH₃)CH₂—.

The term “antagonist,” as used herein, refers to a chemical entity whether a synthesized chemical entity or a natural endogenous chemical entity, that interacts with the glucagon receptor and produces no physiological response of its own, but rather blocks the response to an endogenous or exogenous agonist.

The term “aryl,” as used herein, refers to a monocyclic-ring system or a bicyclic-ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention can be optionally substituted with 0, 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, methylenedioxy, nitro, R₁R₂N—, R₁R₂Nalkyl, R₁R₂Ncarbonyl and R₁R₂Nsulfonyl, wherein R₁ and R₂ are independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “arylaryl,” as used herein, refers to an aryl group, as defined herein, that is appended to the parent molecular moiety through an aryl group, as defined herein.

The term “arylalkoxy,” as used herein, refers to an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl,” as used herein, refers to an arylalkoxy group, as defined herein, appended to the parent molecular moiety through a carbonyl group, as defined herein. Representative examples of arylalkoxycarbonyl include, but are not limited to, benzyloxycarbonyl and naphth-2-ylmethoxycarbonyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkylcarbonyl,” as used herein, refers to an arylalkyl group, as defined herein, appended to the parent molecular moiety through a carbonyl group, as defined herein. Representative examples of arylalkylcarbonyl include, but are not limited to, phenylacetyl and 3-phenylpropanoyl.

The term “arylalkylsulfonyl,” as used herein, refers to an arylalkyl group, as defined herein, appended to the parent molecular moiety through a sulfonyl group, as defined herein. Representative examples of arylalkylsulfonyl include, but are not limited to, benzylsulfonyl and 2-phenylethylsulfonyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, 4-cyanobenzoyl, and naphthoyl.

The term “arylheterocycle,” as used herein, refers to an aryl group, as defined herein, appended to the parent molecular moiety through a heterocycle group, as defined herein.

The term “aryloxy,” as used herein, refers to an aryl group, as defined herein, appended to the parent molecular moiety through an oxy moiety, as defined herein. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “arylsulfonyl,” as used herein, refers to an aryl group, as defined herein, appended to the parent molecular moiety through a sulfonyl group, as defined herein. Representative examples of arylsulfonyl include, but are not limited to, phenylsulfonyl, 4-bromophenylsulfonyl and naphthylsulfonyl.

The term “carbonyl,” as used herein, refers to a —C(O)— group.

The term “carboxy,” as used herein, refers to a —CO₂H group.

The term “carboxyalkenyl,” as used herein, refers to a carboxy group, as defined herein, appended to the parent molecular moiety through an alkenyl group, as defined herein. Representative examples of carboxyalkenyl include, but are not limited to, carboxymethoxy, carboxyethenyl and 3-carboxy-1-propenyl.

The term “carboxyalkoxy,” as used herein, refers to a carboxy group, as defined herein, appended to the parent molecular moiety through an alkoxy group, as defined herein. Representative examples of carboxyalkoxy include, but are not limited to, carboxymethoxy, 2-carboxyethoxy and 3-carboxypropoxy.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “carboxyalkynyl,” as used herein, refers to a carboxy group, as defined herein, appended to the parent molecular moiety through an alkynyl group, as defined herein. Representative examples of carboxyalkynyl include, but are not limited to, carboxymethoxy, carboxyethynyl and 3-carboxy-1-propynyl.

The term “cyano,” as used herein, refers to a —CN group.

The term “cyanoalkoxy,” as used herein, refers to a cyano group, as defined herein, appended to the parent molecular moiety through an alkoxy group, as defined herein. Representative examples of cyanoalkoxy include, but are not limited to, cyanomethoxy, 2-cyanoethoxy and 3-cyanopropoxy.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cyanoalkylthio,” as used herein, refers to a cyanoalkyl group, as defined herein, appended to the parent molecular moiety through a sulfur atom, as defined herein. Representative examples of cyanoalkylthio include, but are not limited to, cyanomethylsulfanyl, 2-cyanoethylsulfanyl and 3-cyanopropylsulfanyl.

The term “cycloalkyl,” as used herein, refers to a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycloalkyl groups of this invention can be substituted with 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, nitro, R₁R₂N—, R₁R₂Nalkyl, R₁R₂Ncarbonyl and R₁R₂Nsulfonyl, wherein R₁ and R₂ are independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “cycloalkylalkoxy,” as used herein, refers to a cycloalkyl group, as defined herein, appended to the parent molecular moiety through an alkoxy group, as defined herein. Representative examples of cycloalkylalkoxy include, but are not limited to, cyclopropylmethoxy, 2-cyclobutylethoxy, 2-cyclopropylethoxy, cyclopentylmethoxy, cyclohexylmethoxy, and 4-cycloheptylbutyl.

The term “cycloalkylalkyl,” as used herein, refers to a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkylalkylcarbonyl,” as used herein, refers to cycloalkylalkyl group, as defined herein, appended to the parent molecular moiety through a carbonyl group, as defined herein. Representative examples of cycloalkylalkylcarbonyl include, but are not limited to, cyclopropylcarbonyl, cyclohexylacetyl and 3-cyclohexylpropanoyl.

The term “cycloalkylalkylsulfonyl,” as used herein, refers to cycloalkylalkyl group, as defined herein, appended to the parent molecular moiety through a sulfonyl group, as defined herein. Representative examples of cycloalkylalkylsulfonyl include, but are not limited to, cyclohexylmethylsulfonyl and 2-cyclohexylethylsulfonyl.

The term “cycloalkylcarbonyl,” as used herein, refers to cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, and cyclohexylcarbonyl.

The term “cycloalkyloxy,” as used herein, refers to cycloalkyl group, as defined herein, appended to the parent molecular moiety through an oxy group, as defined herein. Representative examples of cycloalkyloxy include, but are not limited to, cyclopropyloxy and cyclobutyloxy.

The term “cycloalkylsulfonyl,” as used herein, refers to cycloalkyl group, as defined herein, appended to the parent molecular moiety through a sulfonyl group, as defined herein. Representative examples of cycloalkylsulfonyl include, but are not limited to, cyclohexylsulfonyl and cyclobutylcarbonyl.

The term “ethylenedioxy,” as used herein, refers to a —O(CH₂)₂O— group wherein the oxygen atoms of the ethylenedioxy group are attached to two adjacent carbon atoms of the parent molecular moiety forming a six membered ring.

The term “formyl,” as used herein, refers to a —C(O)H group.

The term “formylalkoxy,” as used herein, refers to a formyl group, as defined herein, appended to the parent molecular moiety through an oxygen atom, as defined herein. Representative examples of formylalkoxy include, but are not limited to, 2-formylethoxy and 3-formylpropoxy.

The term “formylalkyl,” as used herein, refers to a formyl group, as defined herein, appended to the parent molecular moiety through an alkyl group, as defined herein. Representative examples of formylalkyl include, but are not limited to, formylmethyl and 2-formylethyl.

The term “halo” or “halogen,” as used herein, refers to —Cl, —Br, —I or —F.

The term “haloalkenyl,” as used herein, refers to at least one halogen, as defined herein, appended to the parent molecular moiety through an alkenyl group, as defined herein. Representative examples of haloalkenyl include, but are not limited to, 2-bromoethenyl, 3-bromo-2-propenyl and 1-bromo-2-propenyl.

The term “haloalkenyloxy,” as used herein, refers to at least one halogen, as defined herein, appended to the parent molecular moiety through an alkenyloxy group, as defined herein. Representative examples of haloalkenyloxy include, but are not limited to, 3-bromo-2-propenyloxy and 4-bromo-3-butenyloxy.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “haloalkynyl,” as used herein, refers to at least one halogen, as defined herein, appended to the parent molecular moiety through an alkynyl group, as defined herein. Representative examples of haloalkynyl include, but are not limited to, 2-bromoethynyl, 3-bromo-2-propynyl and 1-bromo-2-propynyl.

The term “haloalkynyloxy,” as used herein, refers to at least one halogen, as defined herein, appended to the parent molecular moiety through an alkynyloxy group, as defined herein. Representative examples of haloalkynyloxy include, but are not limited to, (3-bromo-2-propynyl)oxy and (4-bromo-3-butynyl)oxy.

The term “heterocycle” or “heterocyclic,” as used herein, refers to a monocyclic or bicyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered rings have from 0-3 double bonds. Representative examples of monocyclic ring systems include, but are not limited to, azetidinyl, azepinyl, aziridinyl, diazepinyl, 1,3-dioxolanyl, diokanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another heterocyclic monocyclic ring system. Representative examples of bicyclic ring systems include but are not limited to, for example, benzimidazolyl, benzothiazolyl, benzothienyl, benzoxazolyl, benzofuranyl, benzopyranyl, benzothiopyranyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridyl, quinolinyl, quinolizinyl, quinoxalinyl, quinazolinyl, tetrahydroisoquinolinyl, tetrahydroquinolinyl, and thiopyranopyridyl.

The heterocycles of this invention can be substituted with 1, 2 or 3 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, methylenedioxy, nitro, R₁R₂N—, R₁R₂Nalkyl, R₁R₂Ncarbonyl and R₁R₂Nsulfonyl, wherein R₁ and R₂ are independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “heterocyclealkylcarbonyl,” as used herein, refers to a heterocyclealkyl, as defined herein, appended to the parent molecular moiety through a carbonyl group, as defined herein. Representative examples of heterocyclealkylcarbonyl include, but are not limited to, 3-(2-pyrimidinyl)propanoyl and 2-pyridinylacetyl.

The term “heterocyclearyl,” as used herein, refers to a heterocycle, as defined herein, appended to the parent molecular moiety through an aryl group, as defined herein.

The term “heterocyclealkylsulfonyl,” as used herein, refers to a heterocyclealkyl, as defined herein, appended to the parent molecular moiety through a sulfonyl group, as defined herein. Representative examples of heterocyclealkylsulfonyl include, but are not limited to, 4-morpholinylsulfonyl and (2-pyrimidinylmethyl)sulfonyl.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, 1-piperidinylcarbonyl, 4-morpholinylcarbonyl, pyridin-3-ylcarbonyl and quinolin-3-ylcarbonyl.

The term “heterocycleheterocycle,” as used herein, refers to a heterocycle, as defined herein, appended to the parent molecular moiety through a heterocycle group, as defined herein.

The term “heterocyclesulfonyl,” as used herein, refers to a heterocycle, as defined herein, appended to the parent molecular moiety through a sulfonyl group, as defined herein. Representative examples of heterocyclesulfonyl include, but are not limited to, 1-piperidinylcarbonyl, 1-piperidinylsulfonyl, 4-morpholinylsulfonyl, pyridin-3-ylsulfonyl and quinolin-3-ylsulfonyl.

The term “hydroxy,” as used herein, refers to an —OH group.

The term “hydroxyalkoxy,” as used herein, refers to a hydroxy group, as defined herein, appended to the parent molecular moiety through an alkoxy group, as defined herein, wherein the alkyl portion of the alkoxy group is optionally substituted with one or two hydroxy groups. Representative examples of hydroxyalkoxy include, but are not limited to, 2-hydroxyethoxy, 3-hydroxypropoxy and 2-ethyl-4-hydroxyheptyloxy.

The term “hydroxyalkyl,” as used herein, refers to a hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, and 2-ethyl-4-hydroxyheptyl.

The term “inverse agonist,” as used herein, refers to a chemical entity, whether a synthesized chemical entity or a natural endogenous chemical entity, that interacts with the glucagon receptor and functions as an antagonist in non-constitutively active systems but has the added property of actively reducing receptor-mediated constitutive activity. Kenakin, T. FASEB. 15, 598-611, (2001).

The term “mercapto,” as used herein, refers to a —SH group.

The term “mercaptoalkoxy,” as used herein, refers to a mercapto group, as defined herein, appended to the parent molecular moiety through an alkoxy group, as defined herein. Representative examples of mercaptoalkoxy include, but are not limited to, 2-mercaptoethoxy and 3-mercaptopropoxy.

The term “mercaptoalkyl,” as used herein, refers to a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy,” as used herein, refers to a —OCH₂O— group wherein the oxygen atoms of the methylenedioxy are attached to the parent molecular moiety through two adjacent carbon atoms.

The term “nitrogen protecting group” or “N-protecting group,” refers to groups intended to protect an amino group against undesirable reactions during synthetic procedures. Commonly used nitrogen protecting groups are disclosed in T. W. Greene and P. G. M. Wuts, Protective Groups in Organic Synthesis, 3rd edition, John Wiley & Sons, New York (1999). Preferred nitrogen protecting groups are formyl, acetyl, benzoyl, pivaloyl, t-butylacetyl, phenylsulfonyl, benzyl, t-butoxycarbonyl (Boc), and benzyloxycarbonyl (Cbz).

The term “nitro,” as used herein, refers to a —NO₂ group.

The term “oxo,” as used herein, refers to a is O moiety.

The term “sulfinyl,” as used herein, refers to a —S(O)— group.

The term “sulfonyl,” as used herein, refers to a —SO₂— group.

In another embodiment, compounds of the present invention have formula (I) wherein m is 1, n is 1, A is CO₂H, D is phenyl and V, W, X, Y, Z, Rₐ, R_{b}, R_{c}, R_{d}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C(O)—, V is —C(R_{c})—, Y is —(Rₖ)N—, Z is —C(RₚR_{q})— and Rₐ, R_{b}, R_{c}, R_{d}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C(O)—, V is —C(R_{c})—, Y is —(Rₖ)N—, Z is —C(RₚR_{q})—, R_{d} is t-butylphenyl and Rₐ, R_{b}, R_{c}, Rₑ, R_{f}, R_{g}, Rᵢ, Rⱼ, Rₖ, Rₘ, Rₚ, R_{q}, R₅, Rₜ, are defined as in formula (I).

In another embodiment, compounds of the present invention have formula (I) wherein m is 1, n is 1, A is CO₂H, B is H; D is phenyl, W is —(R_{d})N—, X is —C(O)—, V is —C(R_{c})—, Y is —(Rₖ)N—, Z is —C(RₚR_{q})—, R_{d} is selected from the group consisting of cis 4-t-butylcyclohexyl and trans 4-t-butylcyclohexyl and Rₐ, R_{b}, R_{c}, Rₑ, R_{f}, R_{g}, Rᵢ, Rⱼ, Rₖ, Rₘ, Rₚ, R_{q}, R₅, Rₜ, are defined as in formula (I).

In another embodiment, compounds of the present invention have formula (I) wherein m is 1, n is 1, A is CO₂H, B is H; D is phenyl, W is —(R_{d})N—, X is —C═N(Rⱼ)—, V is —C(R)—, Y is —(Rₖ)N—, Z is —C(RₚR_{q})C(RₛRₜ)— and Rₐ, R_{b}, R_{c}, R_{d}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C═N(Rⱼ)—, V is —C(R_{c})—, Y is 0, Z is —C(RₚR_{q})— and Rₐ, R_{b}, R_{c}, R_{d}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C═N(Rⱼ)—, V is —C(R_{c})—, Y is 0, Z is —C(RₚR_{q})—, R_{d} is t-butylphenyl and Rₐ, R_{b}, R_{c}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C═N(Rⱼ)—, V is —C(R_{c})—, Y is O, Z is —C(RₚR_{q})—, R_{d} is selected from the group consisting of cis 4-t-butylcyclohexyl and trans 4-t-butylcyclohexyl and Rₐ, R_{b}, R_{c}, Rₑ, R_{f}, R_{g}, Rᵢ, Rⱼ, Rₖ, Rₘ, Rₚ, R_{q}, Rₛ, Rₜ, are defined as in formula (I).

In another embodiment, compounds of the present invention have formula (I) wherein m is 1, n is 1, A is CO₂H, B is H; D is phenyl, W is —(R_{d})N—, X is —C═N(Rⱼ)—, V is —C(R_{c})—, Y is —(Rₖ)N—, Z is —C(RₚR_{q})— and Rₐ, R_{b}, R_{c}, R_{d}, Rₑ, R_{f}, R_{g}, Rᵢ, Rⱼ, Rₖ, Rₘ, Rₚ, Rₛ, Rₜ, are defined as in formula (I).

Specific compounds of the present invention include, but are not limited to:

N-(4-{3-(4-tert-butylphenyl)-2-oxo-1-[4-(trifluoromethoxy)phenyl]imidazolidin-4-yl}benzoyl)-beta-alanine;

N-(4-{3-(4-tert-butylcyclohexyl)-2-oxo-1-[4-(trifluoromethoxy)phenyl]imidazolidin-4-yl} benzoyl)-beta-alanine;

N-{4-[1-(4-bromophenyl)-3-(4-tert-butylcyclohexyl)-2-oxoimidazolidin-4-yl]benzoyl}-beta-alanine;

N-{4-[3-(4-tert-butylcyclohexyl)-2-oxo-1-(4-phenoxyphenyl)imidazolidin-4-yl]benzoyl}-beta-alanine;

N-[4-((2Z)-3-(4-tert-butylcyclohexyl)-2-{[4-(trifluoromethoxy)phenyl]imino}-1,3-oxazolidin-4-yl)benzoyl]-beta-alanine;

N-{4-[(2Z)-2-[(4-bromophenyl)imino]-3-(4-tert-butylcyclohexyl)-1,3-oxazolidin-4-yl]benzoyl}-beta-alanine;

N-(4-{(2Z)-3-(4-tert-butylcyclohexyl)-2-[(4-phenoxyphenyl)imino]-1,3-oxazolidin-4-yl}benzoyl)-beta-alanine;

N-{4-[1-(4-bromophenyl)-3-(4-tert-butylcyclohexyl)-2-oxoimidazolidin-4-yl]benzoyl}-beta-alanine;

N-{4-[1-(1,1′-biphenyl-4-yl)-3-(4-tert-butylcyclohexyl)-2-oxoimidazolidin-4-yl]benzoyl}-beta-alanine; and

N-{4-[(2Z)-2-(1,1′-biphenyl-4-ylimino)-3-(4-tert-butylcyclohexyl)-1,3-oxazolidin-4-yl]benzoyl}-beta-alanine.

The following additional compounds, representative of formula (I), may be prepared by one skilled in the art using known synthetic methodology or by using synthetic methodology described in the Schemes and Examples contained herein:

3-({4-[3-(4-tert-butylcyclohexyl)-2-oxo-1-phenylimidazolidin-4-yl]benzoyl}amino)propanoic acid;

3-({4-[3-(4-tert-butylcyclohexyl)-1-(2-methylphenyl)-2-oxoimidazolidin-4-yl]benzoyl}amino)propanoic acid;

3-({4-[3-(4-tert-butylcyclohexyl)-1-(3-methylphenyl)-2-oxoimidazolidin-4-yl]benzoyl}amino)propanoic acid;

3-({4-[3-(4-tert-butylcyclohexyl)-1-(4-methylphenyl)-2-oxoimidazolidin-4-yl]benzoyl}amino)propanoic acid;

3-({4-[3-(4-tert-butylcyclohexyl)-1-(2-methoxyphenyl)-2-oxoimidazolidin-4-yl]benzoyl}amino)propanoic acid;

3-({4-[3-(4-tert-butylcyclohexyl)-1-(3-methoxyphenyl)-2-oxoimidazolidin-4-yl]benzoyl}amino)propanoic acid;

3-({4-[3-(4-tert-butylcyclohexyl)-1-(4-methoxyphenyl)-2-oxoimidazolidin-4-yl]benzoyl}amino)propanoic acid;

3-[(4-{3-(4-tert-butylcyclohexyl)-1-[3-(methylthio)phenyl]-2-oxoimidazolidin-4-yl}benzoyl)amino]propanoic acid;

3-({4-[3-(4-tert-butylcyclohexyl)-1-(2-fluorophenyl)-2-oxoimidazolidin-4-yl]benzoyl}amino)propanoic acid;

3-({4-[3-(4-tert-butylcyclohexyl)-1-(3-fluorophenyl)-2-oxoimidazolidin-4-yl]benzoyl}amino)propanoic acid;

3-({4-[3-(4-tert-butylcyclohexyl)-1-(4-fluorophenyl)-2-oxoimidazolidin-4-yl]benzoyl}amino)propanoic acid;

3-({4-[3-(4-tert-butylcyclohexyl)-1-(2-chlorophenyl)-2-oxoimidazolidin-4-yl]benzoyl}amino)propanoic acid;

3-({4-[3-(4-tert-butylcyclohexyl)-1-(3-chlorophenyl)-2-oxoimidazolidin-4-yl]benzoyl}amino)propanoic acid;

3-({4-[3-(4-tert-butylcyclohexyl)-1-(4-chlorophenyl)-2-oxoimidazolidin-4-yl]benzoyl}amino)propanoic acid;

3-({4-[1-(2-bromophenyl)-3-(4-tert-butylcyclohexyl)-2-oxoimidazolidin-4-yl]benzoyl}amino)propanoic acid;

3-({4-[1-(3-bromophenyl)-3-(4-tert-butylcyclohexyl)-2-oxoimidazolidin-4-yl]benzoyl}amino)propanoic acid;

3-({4-[3-(4-tert-butylcyclohexyl)-1-(3-cyanophenyl)-2-oxoimidazolidin-4-yl]benzoyl}amino)propanoic acid;

3-({4-[3-(4-tert-butylcyclohexyl)-1-(4-cyanophenyl)-2-oxoimidazolidin-4-yl]benzoyl}amino)propanoic acid;

3-[(4-{3-(4-tert-butylcyclohexyl)-2-oxo-1-[2-(trifluoromethyl)phenyl]imidazolidin-4-yl}benzoyl)amino]propanoic acid;

3-[(4-{3-(4-tert-butylcyclohexyl)-2-oxo-1-[3-(trifluoromethyl)phenyl]imidazolidin-4-yl}benzoyl)amino]propanoic acid;

3-[(4-{3-(4-tert-butylcyclohexyl)-2-oxo-1-[4-(trifluoromethyl)phenyl]imidazolidin-4-yl}benzoyl)amino]propanoic acid;

3-[(4-{3-(4-tert-butylcyclohexyl)-2-oxo-1-[2-(trifluoromethoxy)phenyl]imidazolidin-4-yl}benzoyl)amino]propanoic acid;

3-({4-[1-(1,3-benzodioxol-5-yl)-3-(4-tert-butylcyclohexyl)-2-oxoimidazolidin-4-yl]benzoyl}amino)propanoic acid;

3-({4-[3-(4-tert-butylcyclohexyl)-1-(2,4-dichlorophenyl)-2-oxoimidazolidin-4-yl]benzoyl}amino)propanoic acid;

3-({4-[1-benzyl-3-(4-tert-butylcyclohexyl)-2-oxoimidazolidin-4-yl]benzoyl}amino)propanoic acid;

3-({4-[1-(3-acetylphenyl)-3-(4-tert-butylcyclohexyl)-2-oxoimidazolidin-4-yl]benzoyl}amino)propanoic acid;

3-({4-[3-(4-tert-butylcyclohexyl)-2-oxo-1-(1-phenylethyl)imidazolidin-4-yl]benzoyl}amino)propanoic acid;

3-[(4-{3-[1-(tert-butoxycarbonyl)piperidin-4-yl]-2-oxo-1-[4-(trifluoromethoxy)phenyl]imidazolidin-4-yl}benzoyl)amino]propanoic acid;

3-[(4-{3-(1-acetylpiperidin-4-yl)-2-oxo-1-[4-(trifluoromethoxy)phenyl]imidazolidin-4-yl}benzoyl)amino]propanoic acid;

3-[(4-{3-(1-methylpiperidin-4-yl)-2-oxo-1-[4-(trifluoromethoxy)phenyl]imidazolidin-4-yl}benzoyl)amino]propanoic acid;

3-[(4-{3-benzyl-2-oxo-1-[4-(trifluoromethoxy)phenyl]imidazolidin-4-yl}benzoyl)amino]propanoic acid;

3-[(4-{3-butyl-2-oxo-1-[4-(trifluoromethoxy)phenyl]imidazolidin-4-yl}benzoyl)amino]propanoic acid;

3-[(4-{3-octyl-2-oxo-1-[4-(trifluoromethoxy)phenyl]imidazolidin-4-yl}benzoyl)amino]propanoic acid;

3-[(4-{3-(cyclohexylmethyl)-2-oxo-1-[4-(trifluoromethoxy)phenyl]imidazolidin-4-yl}benzoyl)amino]propanoic acid;

3-[(4-{3-(4-methylcyclohexyl)-2-oxo-1-[4-(trifluoromethoxy)phenyl]imidazolidin-4-yl}benzoyl)amino]propanoic acid;

3-[(4-{2-oxo-3-(2-phenylethyl)-1-[4-(trifluoromethoxy)phenyl]imidazolidin-4-yl}benzoyl)amino]propanoic acid;

3-[(4-{2-oxo-3-(3-phenylpropyl)-1-[4-(trifluoromethoxy)phenyl]imidazolidin-4-yl}benzoyl)amino]propanoic acid;

3-[(4-{3-(3-ethoxypropyl)-2-oxo-1-[4-(trifluoromethoxy)phenyl]imidazolidin-4-yl}benzoyl)amino]propanoic acid;

3-[(4-{3-(4-tert-butylcyclohexyl)-2-oxo-1-[4-(trifluoromethoxy)phenyl]imidazolidin-4-yl}benzoyl)amino]propanoic acid;

3-[(4-{3-(4-tert-butylcyclohexyl)-2-oxo-1-[4-(trifluoromethoxy)phenyl]imidazolidin-4-yl}benzoyl)amino]propanoic acid;

3-{[4-((2Z)-3-(4-tert-butylcyclohexyl)-2-{[4-(trifluoromethoxy)phenyl]imino}-1,3-oxazolidin-4-yl)benzoyl]amino}propanoic acid;

3-[(4-{(2Z)-3-(4-tert-butylcyclohexyl)-2-[(2-methylphenyl)imino]-1,3-oxazolidin-4-yl}benzoyl)amino]propanoic acid;

3-[(4-{(2Z)-3-(4-tert-butylcyclohexyl)-2-[(3-methylphenyl)imino]-1,3-oxazolidin-4-yl}benzoyl)amino]propanoic acid;

3-[(4-{(2Z)-3-(4-tert-butylcyclohexyl)-2-[(4-methylphenyl)imino]-1,3-oxazolidin-4-yl}benzoyl)amino]propanoic acid;

3-[(4-{(2Z)-3-(4-tert-butylcyclohexyl)-2-[(2-methoxyphenyl)imino]-1,3-oxazolidin-4-yl}benzoyl)amino]propanoic acid;

3-[(4-{(2Z)-3-(4-tert-butylcyclohexyl)-2-[(3-methoxyphenyl)imino]-1,3-oxazolidin-4-yl}benzoyl)amino]propanoic acid;

3-[(4-{(2Z)-3-(4-tert-butylcyclohexyl)-2-[(4-methoxyphenyl)imino]-1,3-oxazolidin-4-yl}benzoyl)amino]propanoic acid;

3-{[4-((2Z)-3-(4-tert-butylcyclohexyl)-2-{[3-(methylthio)phenyl]imino}-1,3-oxazolidin-4-yl)benzoyl]amino}propanoic acid;

3-[(4-{(2Z)-3-(4-tert-butylcyclohexyl)-2-[(2-fluorophenyl)imino]-1,3-oxazolidin-4-yl}benzoyl)amino]propanoic acid;

3-[(4-{(2Z)-3-(4-tert-butylcyclohexyl)-2-[(3-fluorophenyl)imino]-1,3-oxazolidin-4-yl}benzoyl)amino]propanoic acid;

3-[(4-{(2Z)-3-(4-tert-butylcyclohexyl)-2-[(4-fluorophenyl)imino]-1,3-oxazolidin-4-yl}benzoyl)amino]propanoic acid;

3-[(4-{(2Z)-3-(4-tert-butylcyclohexyl)-2-[(2-chlorophenyl)imino]-1,3-oxazolidin-4-yl}benzoyl)amino]propanoic acid;

3-[(4-{(2Z)-3-(4-tert-butylcyclohexyl)-2-[(3-chlorophenyl)imino]-1,3-oxazolidin-4-yl}benzoyl)amino]propanoic acid;

3-[(4-{(2Z)-3-(4-tert-butylcyclohexyl)-2-[(4-chlorophenyl)imino]-1,3-oxazolidin-4-yl}benzoyl)amino]propanoic acid;

3-({4-[(2Z)-2-[(2-bromophenyl)imino]-3-(4-tert-butylcyclohexyl)-1,3-oxazolidin-4-yl]benzoyl}amino)propanoic acid;

3-[(4-{(2Z)-3-(4-tert-butylcyclohexyl)-2-[(3-cyanophenyl)imino]-1,3-oxazolidin-4-yl}benzoyl)amino]propanoic acid;

3-[(4-{(2Z)-3-(4-tert-butylcyclohexyl)-2-[(4-cyanophenyl)imino]-1,3-oxazolidin-4-yl}benzoyl)amino]propanoic acid;

3-{[4-((2Z)-3-(4-tert-butylcyclohexyl)-2-{[2-(trifluoromethyl)phenyl]imino}-1,3-oxazolidin-4-yl)benzoyl]amino}propanoic acid;

3-{[4-((2Z)-3-(4-tert-butylcyclohexyl)-2-{[3-(trifluoromethyl)phenyl]imino}-1,3-oxazolidin-4-yl)benzoyl]amino}propanoic acid;

3-{[4-((2Z)-3-(4-tert-butylcyclohexyl)-2-{[2-(trifluoromethoxy)phenyl]imino}-1,3-oxazolidin-4-yl)benzoyl]amino}propanoic acid;

3-({4-[(2Z)-2-(1,3-benzodioxol-5-ylimino)-3-(4-tert-butylcyclohexyl)-1,3-oxazolidin-4-yl]benzoyl}amino)propanoic acid;

3-[(4-{(2Z)-3-(4-tert-butylcyclohexyl)-2-[(2,4-dichlorophenyl)imino]-1,3-oxazolidin-4-yl}benzoyl)amino]propanoic acid;

3-({4-[(2Z)-2-(benzylimino)-3-(4-tert-butylcyclohexyl)-1,3-oxazolidin-4-yl]benzoyl}amino)propanoic acid;

3-({4-[(2Z)-2-[(3-acetylphenyl)imino]-3-(4-tert-butylcyclohexyl)-1,3-oxazolidin-4-yl]benzoyl}amino)propanoic acid;

3-[(4-{(2Z)-3-(4-tert-butylcyclohexyl)-2-[(2-phenylethyl)imino]-1,3-oxazolidin-4-yl}benzoyl)amino]propanoic acid;

3-({4-[(2Z)-3-(4-tert-butylcyclohexyl)-2-(tert-butylimino)-1,3-oxazolidin-4-yl]benzoyl}amino)propanoic acid;

3-{[(2Z)-3-(4-tert-butylcyclohexyl)-4-(4-{[(2-carboxyethyl)amino]carbonyl} phenyl)-1,3-oxazolidin-2-ylidene]amino}benzoic acid;

3-[(4-{(2Z)-3-(4-tert-butylcyclohexyl)-2-[(1-phenylethyl)imino]-1,3-oxazolidin-4-yl}benzoyl)amino]propanoic acid;

3-({4-[(2Z)-2-[(3-bromophenyl)imino]-3-(4-tert-butylcyclohexyl)-1,3-oxazolidin-4-yl]benzoyl}amino)propanoic acid;

3-{[4-((2Z)-3-(4-tert-butylcyclohexyl)-2-{[4-(trifluoromethyl)phenyl]imino}-1,3-oxazolidin-4-yl)benzoyl]amino}propanoic acid;

3-{[4-((2Z)-3-hexyl-2-{[4-(trifluoromethoxy)phenyl]imino}-1,3-oxazolidin-4-yl)benzoyl]amino}propanoic acid;

3-[(4-{(2Z)-3-(4-tert-butylcyclohexyl)-2-[(4′-methyl-1,1′-biphenyl-4-yl)imino]-1,3-oxazolidin-4-yl}benzoyl)amino]propanoic acid;

3-[(4-{(2Z)-3-(4-tert-butylcyclohexyl)-2-[(3′-chloro-1,1′-biphenyl-4-yl)imino]-1,3-oxazolidin-4-yl}benzoyl)amino]propanoic acid;

3-{[4-((2Z)-3-(4-tert-butylcyclohexyl)-2-{[3′-(trifluoromethyl)-1,1′-biphenyl-4-yl]imino}-1,3-oxazolidin-4-yl)benzoyl]amino}propanoic acid;

3-[(4-{(2Z)-3-(4-tert-butylcyclohexyl)-2-[(4′-phenoxy-1,1′-biphenyl-4-yl)imino]-1,3-oxazolidin-4-yl}benzoyl)amino]propanoic acid;

3-({4-[(2Z)-2-{[4-(1-benzofuran-2-yl)phenyl]imino}-3-(4-tert-butylcyclohexyl)-1,3-oxazolidin-4-yl]benzoyl}amino)propanoic acid;

3-[(4-{(2Z)-3-(4-tert-butylcyclohexyl)-2-[(4′-methyl-1,1′-biphenyl-3-yl)imino]-1,3-oxazolidin-4-yl}benzoyl)amino]propanoic acid;

3-[(4-{(2Z)-3-(4-tert-butylcyclohexyl)-2-[(2′-chloro-1,1′-biphenyl-3-yl)imino]-1,3-oxazolidin-4-yl}benzoyl)amino]propanoic acid;

3-[(4-{(2Z)-3-(4-tert-butylcyclohexyl)-2-[(3′-chloro-1,1′-biphenyl-3-yl)imino]-1,3-oxazolidin-4-yl}benzoyl)amino]propanoic acid;

3-[(4-{(2Z)-3-(4-tert-butylcyclohexyl)-2-[(4′-chloro-1,1′-biphenyl-3-yl)imino]-1,3-oxazolidin-4-yl}benzoyl)amino]propanoic acid;

3-{[4-((2Z)-3-(4-tert-butylcyclohexyl)-2-{[3′-(trifluoromethyl)-1,1′-biphenyl-3-yl]imino}-1,3-oxazolidin-4-yl)benzoyl]amino}propanoic acid;

3-{[4-((2Z)-3-(4-tert-butylcyclohexyl)-2-{[4′-(trifluoromethyl)-1,1′-biphenyl-3-yl]imino}-1,3-oxazolidin-4-yl)benzoyl]amino}propanoic acid;

3-{[4-((2Z)-3-(4-tert-butylcyclohexyl)-2-{[3′-(trifluoromethoxy)-1,1′-biphenyl-3-yl]imino}-1,3-oxazolidin-4-yl)benzoyl]amino}propanoic acid;

3-{[4-((2Z)-3-(4-tert-butylcyclohexyl)-2-{[4′-(trifluoromethoxy)-1,1′-biphenyl-3-yl]imino}-1,3-oxazolidin-4-yl)benzoyl]amino}propanoic acid;

3-[(4-{(2Z)-3-(4-tert-butylcyclohexyl)-2-[(3′,5′-dimethyl-1,1′-biphenyl-3-yl)imino]-1,3-oxazolidin-4-yl}benzoyl)amino]propanoic acid;

3-({4-[(2Z)-2-{[3-(1-benzofuran-2-yl)phenyl]imino}-3-(4-tert-butylcyclohexyl)-1,3-oxazolidin-4-yl]benzoyl}amino)propanoic acid;

3-({4-[(2Z)-2-{[2-(1-benzofuran-2-yl)phenyl]imino}-3-(4-tert-butylcyclohexyl)-1,3-oxazolidin-4-yl]benzoyl}amino)propanoic acid;

3-({4-[(2Z)-2-(1,1′-biphenyl-3-ylimino)-3-(4-tert-butylcyclohexyl)-1,3-oxazolidin-4-yl]benzoyl}amino)propanoic acid;

3-({4-[(2Z)-2-(1,1′-biphenyl-2-ylimino)-3-(4-tert-butylcyclohexyl)-1,3-oxazolidin-4-yl]benzoyl}amino)propanoic acid;

3-{[4-((2Z)-3-[1-(tert-butoxycarbonyl)piperidin-4-yl]-2-{[4-(trifluoromethoxy)phenyl]imino}-1,3-oxazolidin-4-yl)benzoyl]amino}propanoic acid;

3-{[4-((2Z)-3-(1-acetylpiperidin-4-yl)-2-{[4-(trifluoromethoxy)phenyl]imino}-1,3-oxazolidin-4-yl)benzoyl]amino}propanoic acid;

3-{[4-((2Z)-3-(1-methylpiperidin-4-yl)-2-{[4-(trifluoromethoxy)phenyl]imino}-1,3-oxazolidin-4-yl)benzoyl]amino}propanoic acid;

3-{[4-((2Z)-3-benzyl-2-{[4-(trifluoromethoxy)phenyl]imino}-1,3-oxazolidin-4-yl)benzoyl]amino}propanoic acid;

3-{[4-((2Z)-3-butyl-2-{[4-(trifluoromethoxy)phenyl]imino}-1,3-oxazolidin-4-yl)benzoyl]amino}propanoic acid;

3-{[4-((2Z)-3-octyl-2-{[4-(trifluoromethoxy)phenyl]imino}-1,3-oxazolidin-4-yl)benzoyl]amino}propanoic acid;

3-{[4-((2Z)-3-(cyclohexylmethyl)-2-{[4-(trifluoromethoxy)phenyl]imino}-1,3-oxazolidin-4-yl)benzoyl]amino}propanoic acid;

3-{[4-((2Z)-3-(4-methylcyclohexyl)-2-{[4-(trifluoromethoxy)phenyl]imino}-1,3-oxazolidin-4-yl)benzoyl]amino}propanoic acid;

3-{[4-((2Z)-3-(2-phenylethyl)-2-{[4-(trifluoromethoxy)phenyl]imino}-1,3-oxazolidin-4-yl)benzoyl]amino}propanoic acid;

3-{[4-((2Z)-3-(3-phenylpropyl)-2-{[4-(trifluoromethoxy)phenyl]imino}-1,3-oxazolidin-4-yl)benzoyl]amino}propanoic acid;

3-{[4-((2Z)-3-(3-ethoxypropyl)-2-{[4-(trifluoromethoxy)phenyl]imino}-1,3-oxazolidin-4-yl)benzoyl]amino}propanoic acid;

3-{[4-((2Z)-3-(3,3-dimethylbutyl)-2-{[4-(trifluoromethoxy)phenyl]imino}-1,3-oxazolidin-4-yl)benzoyl]amino}propanoic acid compound with N-(3,3-dimethylbutyl)-N′-[4-(trifluoromethoxy)phenyl]urea (1:1);

3-{[4-((2Z)-3-(2-propoxyethyl)-2-{[4-(trifluoromethoxy)phenyl]imino}-1,3-oxazolidin-4-yl)benzoyl]amino}propanoic acid compound with N-(2-propoxyethyl)-N′-[4-(trifluoromethoxy)phenyl]urea (1:1);

3-{[4-((2Z)-3-(4-tert-butylcyclohexyl)-2-{[4-(trifluoromethoxy)phenyl]imino}-1,3-oxazolidin-4-yl)benzoyl]amino}propanoic acid; and

3-{[4-((2Z)-3-(4-tert-butylcyclohexyl)-2-{[4-(trifluoromethoxy)phenyl]imino}-1,3-oxazolidin-4-yl)benzoyl]amino}propanoic acid.

The present compounds can exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I) for example, by hydrolysis in blood.

Asymmetric centers can exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well-known in the art.

Geometric isomers can exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration.

Therapeutic compositions of the present compounds comprise an effective amount of the same formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

The antagonism/inverse agonism of glucagons on glucagons receptor by the compounds of the present invention can be delayed by using a liquid suspension of crystalline or amorphous material with poor water solubility. The rate of absorption of the compounds depends upon their rate of dissolution which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, therapeutically suitable excipient such as a carrier, filler, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient which is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally comprise tableting lubricants and aids. Capsules can also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of the compound of formula (I) to effectively ameliorate disorders by modulated by glucagon on the human glucagon receptor at a reasonable benefit/risk ratio applicable to any medical treatment. The specific therapeutically effective dose level for any particular patient will depend upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to antagonize the action of glucagon on the glucagon receptor in single or divided doses can be in amounts, for example, from about 0.01 mg/kg/day to about 50 mg/kg/day body weight. In a more preferred range, compounds of the present invention necessary to antagonize the action of glucagon on the glucagon receptor in a single or divided doses from about 0.1 mg/kg/day to about 25 mg/kg/day body weight. Single dose compositions can contain such amounts or multiple doses thereof of the compounds of the present invention to make up the daily dose. In general, treatment regimens comprise administration to a patient in need of such treatment from about 10 mg to about 1000 mg of the compounds per day in single or multiple doses.

### Biological Data

Methods for Glucagon-mediated Cyclic AMP Production Studies in Primary Human Hepatocytes

### Preparation of Human Hepatocytes

Primary human hepatocytes (Catalog number: F00986) were purchased from In Vitro Technologies (Baltimore, Md.). Hepatocytes were delivered in a 50 mL conical tube packed on ice. Cells were transferred from the conical tube into a 162 cm² tissue culture flask and placed in a 37° C. incubator under 95% O₂/5% CO₂ atmosphere for 2 hr. Cells were harvested in a 50 mL conical tube and centrifuged at 1000 RPM for 5 min in a Beckman GS-6R centrifuge. The supernatant was discarded and the cell pellet was resuspended in stimulation buffer with isobutylmethylxanthine (Perkin Elmer Life Sciences, Boston, Mass.) at 4.7 million cells per mL.

### Evaluation of Glucagon Antagonists on Glucagon-Stimulated cAMP Production

Glucagon-stimulated cAMP production was determined using the adenylyl cyclase activation Flashplate assay from Perkin-Elmer Life Sciences (Catalog number SMP004B, Boston, Mass.). Stock solutions (10 mM) of glucagon antagonists were prepared by dissolving compounds in appropriate volumes of 100% dimethylsulfoxide. 40 μM solutions were prepared by adding 1.2 μL of the 10 mM stock into 300 μL of Dulbecco's phosphate buffered saline containing 0.008% bacitracin. Introduced into the Flashplate wells were 50 μL of the 40 μM compound (final concentration is 20 μM) and 25 μL of human hepatocyte suspension (approximately 117,000 cells/well). The plates were left at room temperature for 15 min. 25 μL of 60 nM glucagon, prepared in stimulation buffer, were added (final concentration is 15 nM) and the mixture was incubated in a 37° C. incubator for 30 min. Afterward, 100 μL of the detection buffer mixed with [¹²⁵I]-succinyl cAMP tyrosine methyl ester (Perkin-Elmer Life Sciences, Boston, Mass.) were added, and the mixture was incubated at room temperature for an additional 2 hr. The contents were then aspirated and the amount of [¹²⁵I]-cAMP bound to the plate was measured by TopCount (Perkin-Elmer Life Sciences, Boston, Mass.). Quantitation of cAMP was calculated from a cAMP standard curve.

The compounds of the present invention were found to inhibit glucagon-stimulated cAMP production at a concentration of 20 μM in a range of about 50% to about 100%. In a preferred range, the compounds of the present invention were found to inhibit glucagon-stimulated cAMP production at a concentration of 20 μM in a range of about 85% to about 100%. As inhibitors of glucagon-stimulated cAMP production, the compounds of the present invention demonstrate their utility in the antagonism/inverse agonism of the human glucagon receptor. Therefore, the data provided indicates that the compounds of the present invention are antagonists/inverse agonists of the human glucagon receptor and may be useful for the treatment of type 2 diabetes and related metabolic disorders including but not limited to hyperglycemia, inadequate glucose clearance, obesity, hyperinsulinemia, type 1 diabetes, Syndrome X, and hypertriglyceridemia.

For the treatment of diabetes or Syndrome X, compounds of the present invention may be used alone, or in combination with any existing anti-diabetic agent. Agents which may be used in combination with the compounds of the present invention include, but are not limited to insulin, an insulin analog such as mecasermin and the like, an insulin secretagogue such as nateglinide and the like, a biguanide such as metformin and the like, a sulfonylurea such chlorpropamide, glipizide, glyburide and the like, an insulin sensitizing agent such as troglitazone, pioglitazone, rosiglitazone and the like, an α-glucosidase inhibitor such as acarbose, voglibose, miglitol and the like, an aldose reductase inhibitor such as zopolrestat and the like, a metiglinide such as repaglinide and the like, or a glycogen phosphorylase inhibitor. Other such anti-diabetic agents are known to one skilled in the art. The ability of the compounds of the present invention to treat diabetes, alone or in combination with another agent, can be demonstrated according to the methods described by Friedman, J. E., Y. Sun, T. Ishizuka, C. J. Farrell, S. E. McCormack, L. M. Herron, P. Hakimi, P. Lechner, and J. S. Yun, in J. Biol. Chem. 272 (50): 31475-31481, 1997; or, according to the methods described herein.

For the treatment of obesity, compounds of the present invention may be used alone, or in combination with any existing anti-obesity agent. Agents which may be used in combination with the compounds of the present invention include, but are not limited to fatty acid uptake inhibitors such as orlistat and the like, monoamine reuptake inhibitors such as sibutramine and the like, anorectic agents such as dexfenfluramine, bromocryptine and the like, sympathomimetics such as phentermine, phendimetrazine, mazindol and the like, or thyromimetic agents. Other such anti-obesity agents are known to one skilled in the art. The ability of the compounds of the present invention to treat obesity, alone or in combination with another agent, can be demonstrated according to the methods described by Walker, H. C., and D. R. Romsos, in Am. J. Physiol. 262 (Endocrinol. Metab. 25): E110-E117, 1992; or according to the methods described by Langley, S. C., and D. A. York, in Am. J. Physiol. 259 (Regulaory Integrative Comp. Physiol. 28): R539-R544, 1990.

### Abbreviations

Abbreviations which have been used in the descriptions of the schemes and the examples that follow are: BF₃.OEt₂ for boron trifluoride diethyl ether complex; DMF for N,N-dimethylformamide; DMSO for dimethylsulfoxide; EtOAc for ethyl acetate; EtOH for ethanol; MeOH for methanol; Ms for mesylate or —S(O)₂CH₃; Tf for triflate or —S(O)₂CF₃; THF for tetrahydrofuran; and Ts for tosylate or —S(O)₂-(para-CH₃Ph).

### Preparation of Compounds of the Invention

The compounds and processes of the present invention will be better understood in connection with the following synthetic schemes and methods which illustrate a means by which the compounds of the invention can be prepared.

The compounds of this invention can be prepared by a variety of procedures and synthetic routes. Representative procedures and synthetic routes are shown in, but are not limited to, Scheme 1.

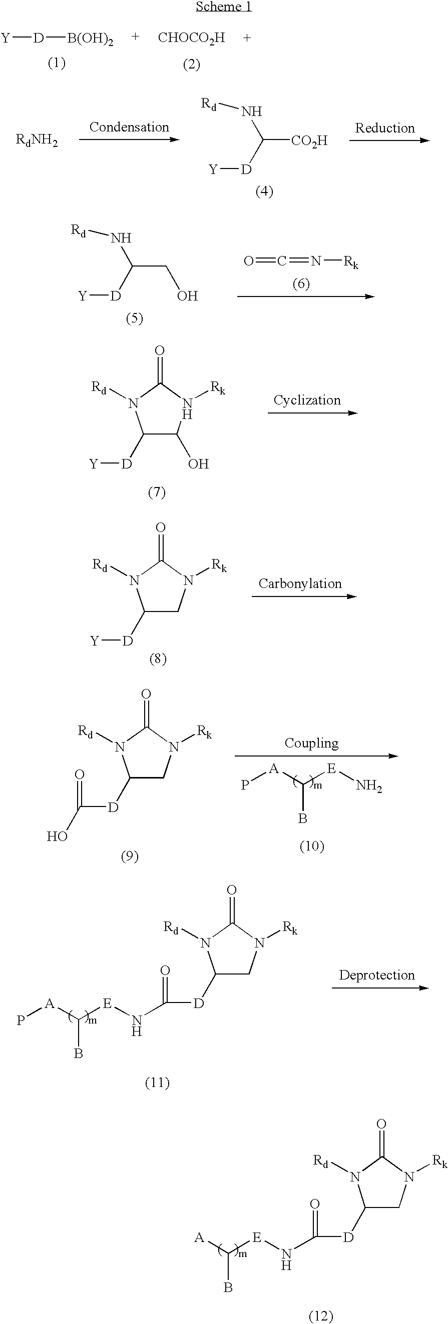

Amides of general formula (12), wherein A, B, D, E and m are defined as in formula (I) and R_{d} and Rₖ are defined in formula (I), may be prepared as shown in scheme 1. Boronic acids of general formula (1), wherein Y is a halide like bromine and D is defined as in formula (1) may be purchased or prepared using methodology known to those skilled in the art. Boronic acids of general formula (1) may be treated with aldehydes like glyoxylic acid monohydrate (2) and amines (3) to provide amino acids of general formula (4). Amines (3) and amino acids (4) may be purchased or prepared using methodology known to those in the art. Acids of general formula (4) may be reduced to provide alcohols of general formula (5) by hydrolysis to the corresponding ester follwed by reduction with sodium borohydride or other methods known to those skilled in the art. Alcohols of general formula (5) may be acylated with isocyanates of general formula (6) to provide ureas of general formula (7). Isocyanates (6) may be purchased or prepared by methods known to those skilled in the art. Ureas of general formula (7) may be cyclized with thionyl chloride and lithium chloride or using methodology known to those skilled in the art to provide cyclic ureas of general formula (8). Carbonylation of halide (8) using a palladium catalyst like [1,1′-bis(diphenylphosphino)ethane]ferrocene]dichloropalladium(II) complex with dichloromethane (1:1), a base like triethylamine, and an alcohol like methanol provides esters which can be hydrolyzed with a base like sodium hydroxide to provide acids (9). Acids (9) can be coupled with protected amines (10) or their salts using 1-(3-dimethylaminopropyl)-3-ethylcarbodiimide hydrochloride and 1-hydroxybenzotriazole hydrate or other reagents known to those skilled in the art to provide amides (11). Protected amines (10) can be purchased or prepared by methods known to those skilled in the art. Protected amides (11) can be deprotected by methods known to those skilled in the art to yield amides of general formula (12).

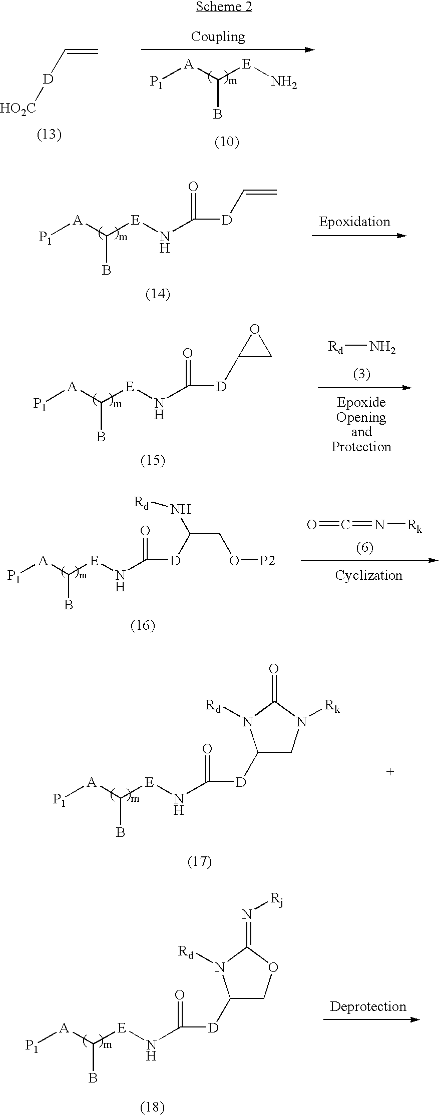

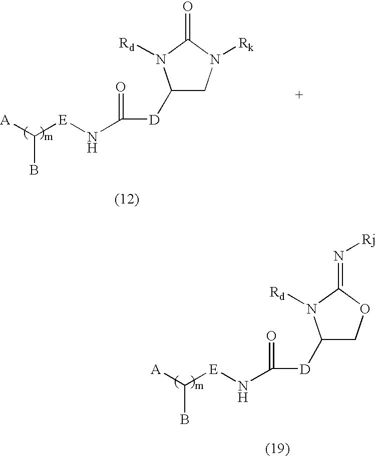

Amides of general formula (12) and (18), wherein A, B, D, E and m are defined as in formula (I) and R_{d} and Rₖ are defined in formula (I), may be prepared as shown in scheme 2. Styrenes of general formula (13), wherein D is defined as in formula (1) may be purchased or prepared using methodology known to those skilled in the art. Styrenes of general formula (13) may be treated with protected amines (10) or their salts like β-alanine ethyl ester hydrochloride using 1-(3-dimethylaminopropyl)-3-ethylcarbodiimide hydrochloride and 1-hydroxybenzotriazole hydrate or other reagents known to those skilled in the art to provide amides of general formula (14). Amides of general formula (14) may be epoxidized with 3-chloroperbenzoic acid or other methods known to those skilled in the art to provide epoxides of general formula (15). Epoxides of general formula (15) may be opened with amines of general formula (3) to provide alcohols which can be protected using silylating reagents like t-butyldimethylsilylchloride to provide protected amino alcohols of general formula (16). Amines of general formula (16) may be reacted with isocyanates (6) which can be purchased or prepared by methods known to those skilled in the art. The resulting ureas can then be deprotected, for example with tetrabutylammonium fluoride to remove a silyl protecting group, and cyclized with diethyl azodicarboxylate and polymer supported triphenylphosphine, a base like potassium t-butoxide and polymer supported p-toluenesulfonyl chloride, or using methodology known to those skilled in the art to provide cyclic ureas of general formula (17) and/or heterocycles of general formula (18). Protected cyclic ureas (11) and/or protected heterocycles (18) can be deprotected by methods known to those skilled in the art to yield acids of general formula (12) and/or (19). In some cyclization protocols, like treatment with potassium t-butoxide and polymer supported p-toluenesulfonyl chloride, deprotection occurs during the reaction allowing for the synthesis of acids (12) and/or (19) without a deprotection step.

The compounds and processes of the present invention will be better understood by reference to the following examples, which are intended as an illustration of and not a limitation upon the scope of the invention. Further, all citations herein are incorporated by reference.

Compounds of the invention were named by ACD/ChemSketch version 5.01 (developed by Advanced Chemistry Development, Inc., Toronto, ON, Canada) or were given names consistent with ACD nomenclature.

### EXPERIMENTALS

### Example 1

### N-(4-{3-(4-tert-butylphenyl)-2-oxo-1-[4-(trifluoromethoxy)phenyl]imidazolidin-4-yl}benzoyl)-beta-alanine

### Example 1A

### (4-Bromo-phenyl)-(4-tert-butyl-phenylamino)-acetic acid methyl ester

A solution of 4-tert-butylaniline (3.01 g, 20.18 mmol, purchased from Aldrich) and glyoxylic acid monohydrate (1.857 g, 20.18 mmol, purchased from Aldrich) was stirred vigorously in dichloroethane (150 mL) for 1 hour. 4-bromophenylboronic acid (4.05 g, 20.15 mmol, purchased from Aldrich) was then added and the reaction mixture heated to 50° C. for 36 hours. The solvent was evaporated under reduced pressure and the residue dissolved in saturated anhydrous HCl in methanol (50 mL) and heated to 60° C. for 2 hours. The solvent was evaporated under reduced pressure and the residue partitioned between ethyl acetate (80 mL) and saturated sodium bicarbonate (30 mL). The organic layer was separated, washed with brine (30 mL), dried (MgSO₄) and solvent evaporated under reduced pressure. The crude oil was purified by flash chromatography (silica gel, 100:0→90:10 hexanes:ethyl acetate) to provide the title compound as a light brown solid (0.775 g, 10%).

### Example 1B

### 2-(4-Bromo-phenyl)-2-(4-tert-butyl-phenylamino)-ethanol

The product from Example 1A (0.5 g, 1.33 mmol) was dissolved in ethanol (15 mL) and solid sodium borohydride (0.253 g, 6.66 mmol) was added and the reaction stirred at ambient for 12 hours. The reaction was cooled to 0° C. (ice/water) and glacial acetic acid (5 mL) was added dropwise and stirred for 1 hour. The solvent was evaporated under reduced pressure and the residue partitioned between methylene chloride (100 mL) and saturated sodium bicarbonate (30 mL. The organic layer was separated, dried (MgSO₄) and solvent evaporated under reduced pressure. The crude product was purified by flash column chromatography (silica gel, 100:0→70:30 hexanes:ethyl acetate) to provide the title compound as a white solid (0.425 g, 92%).

### Example 1C

### 4-(4-Bromo-phenyl)-3-(4-tert-butyl-phenyl)-1-(4-trifluoromethoxy-phenyl)-imidazolidin-2-one

The product from Example 1B (0.334 g, 0.96 mmol) was dissolved in THF (2.5 mL) and added 4-(trifluoromethoxy)phenyl isocyanate (0.195 g, 0.96 mmol, purchased from Aldrich) and stirred at ambient temperature for 48 hours. The solvent was evaporated under reduced pressure and the product purified by flash column chromatography (silica gel, 100:0→80:20 hexanes:ethyl acetate) to collect an oil. This intermediate was dissolved in CH₂Cl₂ (5 mL) and added lithium chloride (0.043 g, 1.03 mmol), thionyl chloride (0.123 g, 1.03 mmol) and stirred at ambient temperature for two hours. The reaction was quenched with saturated ammonium chloride (3 mL) and extracted with ethyl acetate (15 mL). The organic layer was separated, dried (MgSO₄), filtered and evaporated to dryness under reduced pressure. The residue solid was dissolved in THF (5 mL) and added dropwise to a solution of sodium hydride (0.055 gm, 2.2 mM) in THF (3 mL) at 0° C. The reaction was stirred for 1 hour and quenched with saturated ammonium chloride (2 mL) and extracted with ethyl acetate (15 mL), dried (MgSO4), filtered and solvent evaporated under reduced pressure. The product was purified by flash column chromatography (silica gel, 100:0→85:15 hexanes:ethyl acetate) to provide the title compound as a white solid (0.2 g, 74%).

### Example 1D

### 4-[3(4-tert-Butyl-phenyl)-2-oxo-1-(4-trifluoromethoxy-phenyl)-imidazolidin-4-yl]-benzoic acid methyl ester

To a solution of product from Example 1C (0.2 g, 0.37 mmol)) in MeOH (10 mL) was added triethyl amine (0.2 g, 2.0 mmol), PdCl₂(dppf)-CH₂Cl₂ (0.031 g, 0.04 mmol) and heated to 120° C. with carbon monoxide (600 psi) for 16 hours. The reaction was filtered and solvent evaporated under reduced pressure followed by purification with flash column chromatography (silica gel, 100:0→70:30 hexanes:ethyl acetate) to provide the title compound as a white solid (0.179 g, 95%).

### Example 1E

The product from Example 1D (0.055 g, 0.1 mmol) was dissolved in 1:1 MeOH/water (1.5 mL) and added sodium hydroxide (0.01 g, 0.25 mmol) and stirred for 12 hours. The reaction was neutralized with solid ammonium chloride (0.03 g, 0.5 mmol) and solvent evaporated under reduced pressure. The residue was dissolved in DMF (1.5 mL) and added 1-(3-dimethylaminopropyl)-3-ethylcarbodiimide.HCl (0.031 g, 0.16 mmol), hydroxybenzotriazole hydrate (0.021 g, 0.16 mmol), β-alanine ethyl ester.HCl (0.024 g, 0.016 mmol) and di-isopropylethyl amine (0.058 mg, 0.3 mmol) and stirred at ambient temperature for 12 hours. The reaction was partitioned between methylene chloride (10 ml) and saturated sodium bicarbonate (2 ml) and the organic layer separated and solvent evaporated under reduced pressure. The residue was dissolved in 1:1 MeOH/water (1.5 mL) and added sodium hydroxide (0.03 g, 0.7 mmol) and stirred for 12 hours. The reaction was quenched with solid ammonium chloride (0.13 g, 2.4 mmol) and solvent evaporated under reduced pressure. The residue was dissolved in 3:1 acetonitrile/water (3 mL) and purified by reverse phase preparative HPLC (20:80→100:0 acetonitrile/0.1% TFA in water) on a YMC ODS Guardpak column to provide the title compound as a white solid (0.020 g, 35%). ¹H NMR (300 MHz, DMSO-D₆) δ 8.48 (t, J is 4.41, 1H), 7.77 (dd, J is 8.47, 4.41, 4H), 7.5 (d, J is 8.5, 2H), 7.32 (dd, J is 7.46, 4H), 7.27 (dd, J is 8.8, 2H) 5.68 (m, 1H), 4.45 (t, J is 8.6, 1H), 3.70 (m, 1H), 3.2 (m, 2H), 2.25 (m, 2H), 1.1 (s, 9H); MS (APCI+) m/z 570 (M+H)⁺.

### Example 2

### N-(4-{3-(4-tert-butylcyclohexyl)-2-oxo-1-[4-(trifluoromethoxy)phenyl]imidazolidin-4-yl}benzoyl)-beta-alanine

### Example 2A

### 3-(4-Vinyl-benzoylamino)-propionic acid ethyl ester

To a solution of 4-vinylbenzoic acid (1.5 g, 10.12 mmol, purchased from Aldrich) in DMF (10 mL) was added α-alanine ethyl ester.HCl (1.85 g, 12.14 mmol), hydroxybenzotriazole (2.03 g, 15.8 mmol), 1-(3-dimethylaminopropyl)-3-ethylcarbodiimide.HCl (2.92 g, 15.18 mmol) and diisopropylethyl amine (4.56 g, 35.4 mmol) and stirred for 12 hours. The reaction was partitioned between water (10 mL) and ethyl acetate (100 mL). The organic layer was separated, washed once with brine (20 mL), dried (MgSO₄), filtered and solvent evaporated under reduced pressure. The product was purified by flash column chromatography (silica gel, 100:0→50:50 hexanes:ethyl acetate) to provide the title compound as a white solid (1.83 g, 73%).

### Example 2B

### 3-(4-Oxiranyl-benzoylamino)-propionic acid ethyl ester

The product from Example 2A was dissolved in chloroform (100 mL) and added meta-chloroperbenzoic acid (2.04 g, 11.85 mmol) and stirred for 12 hours. Added more chloroform (100 mL) and the organic phase was washed twice with 5% sodium bicarbonate (25 mL), then with 5% KI (10 mL). Sufficient 10% sodium thiosulfate (50 mL) was added to decolorize the solution. The organic phase was separated, washed twice with 5% sodium bicarbonate (30 mL), then brine (30 mL), dried (MgSO₄) and solvent evaporated under reduced pressure to provide title compound which did not necessitate further purification (2.3 g, 95%).

### Example 2C

### 3-[4-(1-Bromo-2-hydroxy-ethyl)-benzoylamino]-propionic acid ethyl ester

Triphenylphosphine-HBr (4.44 g, 12.96 mmol) was dissolved in CH₂Cl₂ (100 mL) and cooled to −72° C. (dry ice/acetone). The product from Example 2B (3.1 g, 11.8 mmol) was dissolved in CH₂Cl₂ (20 mL) and added dropwise to the reaction and stirred for 1 hour. The reaction was warmed to 0° C. and added saturated sodium bicarbonate (30 mL). The organic phase was separated, dried (MgSO₄), filtered and solvent evaporated under reduced pressure at 20° C. The crude product was purified by flash column chromatography (silica gel, 100:0→95:5 CH₂Cl₂:MeOH) to provide the title compound as an oil (3.8 g, 93%).

### Example 2D

### 3 {4-[1-Bromo-2-(tert-butyl-dimethyl-silanyloxy)-ethyl]-benzoylamino}-propionic acid ethyl ester

The product from Example 2C (3.8 g, 11.1 mmol) was dissolved in THF (20 mL) cooled to −10° C. (with ice/MeOH) and added imidazole (0.94 g, 13.8 mmol) in THF (5 mL) followed by tert-butyldimethylsilyl chloride (2.0 gm, 13.3 mmol) in THF (7 mL). The reaction was stirred for 12 hours at ambient temperature and added saturated ammonium chloride (15 mL) and extracted with ethyl acetate (75 mL). The organic phase was washed with brine (20 mL), dried (MgSO₄) and evaporated under reduced pressure. The crude product was purified by flash column chromatography (silica gel, 100:0→50:50 hexanes:ethyl acetate) to provide the title compound as a viscous oil (3.4 g, 66%).

### Example 2E

### 3-{4-[1-(4-tert-Butyl-cyclohexylamino)-2-(tert-butyl-dimethyl-silanyloxy)-ethyl]-benzoyl amino}-propionic acid ethyl ester

The product from Example 2D (3.4 g, 7.4 mmol) and tert-butylcyclohexyl amine (2.88 g, 18.5 mmol, purchased from TCI-US, trans/cis mixture of 3.4:1 as determined by ¹H NMR) was dissolved in DMSO (7.5 mL) and heated at 80° C. for 3 hours. The reaction was diluted with 75 ml of ethyl acetate and water (15 mL). The organic layer was separated, washed with brine (25 mL), dried (MgSO₄) and the solvent evaporated under reduced pressure. The crude product was purified by flash column chromatography (silica gel, 100:0→50:50 hexanes:ethyl acetate) to provide title compound as a viscous oil (3.65 g, 91%).

### Example 2F

### 3-{4-[1-[1-(4-tert-Butyl-cyclohexyl)-3-(4-trifluoromethoxy-phenyl)-ureido]-2-(tert-butyl-dimethyl-silanyloxy)-ethyl]-benzoylamino}-propionic acid ethyl ester

The product from Example 2E (0.262 g, 0.5 mmol) was dissolved in THF (1.5 mL) and 4-(trifluoromethoxy)phenyl isocyante (0.1 g, 0.5 mmol) was added and stirred for 12 hours at ambient temperature. The solvent was evaporated under reduced pressure and product purified by flash column chromatography (silica gel, 100:0→65:35 hexanes;ethyl acetate) to provide the title compound as an oil (0.37 g, 99%).

### Example 2G

The product from Example 2F (0.27 g, 0.37 mmol) was dissolved in THF (3 mL) and cooled to 0° C. (ice/water). A solution of tetrabutylammonium fluoride (0.56 mL, 0.56 mmol) in THF was added dropwise and reaction stirred for 30 minutes. The reaction was partitioned between ethyl acetate (15 mL) and saturated ammonium chloride (4 mL). The organic phase was separated, dried (MgSO₄) and purified by flash column chromatography (silica gel, 100:0→50:50 hexanes:ethyl acetate). The purified intermediate (0.182 g, 0.29 mmol) was dissolved in THF (1.5 mL) and added polymer supported triphenylphosphine (0.146 g, 0.44 mmol) followed by diethyl azodicarboxylate (0.076 g, 0.44 mmol) and stirred at ambient temperature for 12 hours. The reaction was filtered and partitioned between ethyl acetate (5 mL) and brine (1 mL). The organic phase was separated and solvent evaporated under reduced pressure. The residue was dissolved in 1:1 MeOH/water (1.5 mL) and added solid sodium hydroxide (0.035 g, 0.87 mmol) and stirred for 3 hours. The reaction was neutralized with 1N HCl (1 mL) and solvent evaporated to dryness. The residue was dissolved in 3:1 acetonitrile/water (3 mL) and purified by reverse phase preparative HPLC (20:80→100:0 acetonitrile/0.1 TFA in water) on a YMC ODS Guardpak column to provide the title compound as a white solid (0.023 g, 11%). ¹H NMR (300 MHz, CDCl₃) δ 7.8 (m, 2H), 7.57 (m, 2H), 7.48 (d, J is 8.5, 1H), 7.37 (d, J is 8.5, 1H), 7.19 (m, 2H), 6.8 (m, 1H), 4.8 (m, 1H), 4.16 (m, 1H), 3.75 (m, 2H), 3.5 (m, 1H), 2.78 (m, 2H), 2.6 (m, 1H) 1.5 (br, 9H), 0.79 (s, 9H); MS (ESI+) m/z 576 (M+H)⁺.

### Example 3

### N-{4-[1-(4-bromophenyl)-3-(4-tert-butylcyclohexyl)-2-oxoimidazolidin-4-yl]benzoyl}-beta-alanine

### Example 3A

### 3-{4-[1-3-(4-Bromo-phenyl)-1-(4-tert-Butyl-cyclohexyl)-ureidol-2-(tert-butyl-dimethyl-silanyloxy)-ethyl]-benzoylamino}-propionic acid ethyl ester

The product from Example 2E (0.23 g, 0.43 mmol) was dissolved in THF (1.5 mL) and 4-bromophenyl isocyanate (0.085 g, 0.43 mmol) was added and reaction stirred for 3 hours. The solvent was evaporated under reduced pressure to provide the title compound as an oil (0.32 g, 99%).

### Example 3B

The product from Example 3A (0.32 g, 0.43 mmol) was dissolved in THF (1.5 mL) and cooled to 0° C. (ice/water). A solution of tetrabutylammonium fluoride (0.65 mL, 0.65 mmol) in THF was added dropwise and reaction stirred for 30 minutes. The reaction was partitioned between ethyl acetate (15 mL) and saturated ammonium chloride (4 mL). The organic phase was separated, dried (MgSO₄) and purified by flash column chromatography (silica gel, 100:0→50:50 hexanes;ethyl acetate). The purified intermediate (0.16 g, 0.26 mmol) was dissolved in THF (3 mL) and cooled to 0° C. (ice/water). Added solid potassium tert-butoxide (0.069 g, 0.62 mmol) followed by polymer supported 4-toluenesulfonyl chloride (0.2 g, 0.31 mmol) and stirred for 90 minutes. The reaction was quenched with water (1 ml) and stirred for a further 1 hour. The reaction was filtered and solid ammonium chloride (0.065 g, 1.2 mmol) was added and stirred for 30 minutes. The solvent was evaporated under reduced pressure and crude product purified by reverse phase preparative HPLC (20:80→100:0 acetonitrile:0.1% TFA in water) to provide the title compound as a white solid (0.037 g, 25%). ¹H NMR (300 MHz, CDCl₃) δ 7.8 (t, 2H), 7.2 (m, 6H), 6.9 (m, 1H), 4.8 (m, 1H), 4.16 (m, 1H), 3.75 (m, 2H), 3.5 (m, 1H), 2.75 (m, 2H), 2.6 (m, 1H) 1.5 (br, 9H), 0.79 (s, 9H); MS (ESI+) m/z 570 (M+H)⁺.

### Example 4

### N-{4-[3-(4-tert-butylcyclohexyl)-2-oxo-1-(4-phenoxyphenyl)imidazolidin-4-yl]benzoyl}-beta-alanine

### Example 4A

### 3-{4-[1-[1-(4-tert-Butyl-cyclohexyl)-3-(4-phenoxy-phenyl)-ureidol-2-(tert-butyl-dimethyl-silanyloxy)-ethyl]-benzoylamino}-propionic acid ethyl ester

The product from Example 2E (0.125 g, 0.23 mmol) was dissolved in THF (1.5 mL) and added 4-phenoxyphenyl isocyanate (0.05 g, 0.23 mmol) and stirred for 12 hours. The solvent was evaporated under reduced pressure to provide the title compound as an oil (0.175 g, 99%).

### Example 4B

The product from Example 4A (0.175 g, 0.23 mmol) was dissolved in THF (1.5 mL) and cooled to 0° C. (ice/water). A solution of tetrabutylammonium fluoride (0.345 mL, 0.345 mmol) in THF was added dropwise and reaction stirred for 30 minutes. The reaction was partitioned between ethyl acetate (15 mL) and saturated ammonium chloride (4 mL). The organic phase was separated, dried (MgSO₄) and purified by flash column chromatography (silica gel 100:0→50:50 hexanes:ethyl acetate). The purified intermediate (0.054 g, 0.085 mmol) was dissolved in THF (3 mL) and cooled to 0° C. (ice/water). Added solid potassium tert-butoxide (0.023 g, 0.21 mmol) followed by polymer supported 4-toluenesulfonyl chloride (0.07 g, 0.1 mmol) and stirred for 90 minutes. The reaction was quenched with water (1 ml) and stirred for a further 1 hour. The reaction was filtered and solid ammonium chloride (0.02 g, 0.3 mmol) was added and stirred for 30 minutes. The solvent was evaporated under reduced pressure and crude product purified by reverse phase preparative HPLC (20:80→100:0 acetonitrile:0.1% TFA in water) on a YMC ODS Guardpak to provide the title compound as a white solid (0.007 g, 14%). ¹H NMR (300 MHz, CDCl₃) δ 7.8 (t, J is 8.3, 2H)), 7.52 (m, 4H), 7. (m, 6H), 6.8 (m, 1H), 4.8 (m, 1H), 4.19 (m, 1H), 3.75 (m, 2H), 3.6 (b, 1H), 3.5 (m, 1H), 2.78 (m, 2H), 2.6 (m, 1H) 1.5 (br, 9H), 0.79 (s, 9H); MS (ESI+) m/z 584 (M+H)⁺.

### Example 5

### N-[4-((2Z)-3-(4-tert-butylcyclohexyl)-2-{[4-(trifluoromethoxy)phenyl]imino}-1,3-oxazolidin-4-yl)benzoyl]-beta-alanine

### Example 5A

The product from Example 2F (0.27 g, 0.37 mmol) was dissolved in THF (3 mL) and cooled to 0 C (ice/water). A solution of tetrabutylammonium fluoride (0.56 mL, 0.56 mmol) in THF was added dropwise and reaction stirred for 30 minutes. The reaction was partitioned between ethyl acetet (15 mL) and saturated ammonium chloride (4 mL). The organic phase was separated, dried (MgSO₄) and purified by flash column chromatography (silica gel, 100:0→50:50 hexanes:ethyl acetate). The purified intermediate (0.182 g, 0.29 mmol) was dissolved in THF (1.5 mL) and added polymer supported triphenylphosphine (0.146 g, 0.44 mmol) followed by diethyl azodicarboxylate (0.076 g, 0.44 mmol) and stirred at ambient temperature for 12 hours. The reaction was filtered and partitioned between ethyl acetate (5 mL) and brine (1 mL). The organic phase was separated and solvent evaporated under reduced pressure. The residue was dissolved in 1:1 MeOH/water (1.5 mL) and added solid sodium hydroxide (0.035 g, 0.87 mmol) and stirred for 3 hours. The reaction was neutralized with 1N HCl (1 mL) and solvent evaporated to dryness under reduced pressure. The residue was dissolved in 3:1 acetonitrile/water (3 mL) and purifed by reverse phase preparative HPLC (20:80→100:0 acetonitrile:0.1% TFA in water) on a YMC ODS Guardpak column to provide the title compound as a white solid (0.058 g, 27%). ¹H NMR (500 MHz, DMSO-d₆) δ 8.41 (t, J is 4.3 Hz, 1H) 7.85 (d, J is 8.3 Hz, 2H), 7.57 (d, J is 8.4 Hz, 2H), 7.31 (m, 4H), 5.29 (m, 1H), 4.85 (t, J is 9.6 Hz, 1H), 4.3 (m, 1H), 3.8 (m, 2H), 2.50 (m, 2H), 2.0 (m, 1H), 1.8 (m, 1H), 1.6 (m, 2H), 1.2 (b, 5H), 0.79 (s, 9H); MS (APCI+) m/z 576 (M+H)⁺.

### Example 6

### N-{4-[(2Z)-2-[(4-bromophenyl)imino]-3-(4-tert-butylcyclohexyl)-1,3-oxazolidin-4-yl]benzoyl}-beta-alanine

### Example 6A

The product from Example 3A (0.32 g, 0.43 mmol) was dissolved in THF (1.5 mL) and cooled to 0° C. (ice/water). A solution of tetrabutylammonium fluoride (0.65 mL, 0.65 mmol) in THF was added dropwise and reaction stirred for 30 minutes. The reaction was partitioned between ethyl acetate (15 mL) and saturated ammonium chloride (4 mL). The organic phase was separated, dried (MgSO₄) and purified by flash column chromatography (silica gel, 1 00:0→50:50 hexanes:ethyl acetate). The purified intermediate (0.16 g, 0.26 mmol) was dissolved in THF (3 mL) and cooled to 0° C. (ice/water). Added solid potassium tert-butoxide (0.069 g, 0.62 mmol) followed by polymer supported 4-toluenesulfonyl chloride (0.2 g, 0.31 mmol) and stirred for 90 minutes. The reaction was quenched with water (1 ml) and stirred for a further 1 hour. The reaction was filtered and solid ammonium chloride (0.065 g, 1.2 mmol) was added and stirred for 30 minutes. The solvent was evaporated under reduced pressure and crude product purified by reverse phase preparative HPLC (20:80→100:0 acetonitrile:0.1% TFA in water) on a YMC ODS Guardpak column to provide the title compound as a white solid (0.028 g, 19%). ¹H NMR (300 MHz, CDCl₃) δ 7.8 (s, 1H), 7.62 (d, J is 8.3 Hz, 2H), 7.40 (d, J is 8.59, 2H), 7.27 (d, J is 8.4 Hz, 2H), 7.19 (d, J is 8.7 Hz, 2H), 4.72 (br, 1H), 4.5 (m, 1H), 4.16 (d, J is 9 Hz, 1H), 3.95 (br 1H), 3.65 (m, 2H), 2.63 (t, J is 7 Hz, 2H), 1.5 (br, 9H), 0.8 (s, 9H); MS (ESI+) m/z 571 (M+H)⁺.

### Example 7

### N-(4-{(2Z)-3-(4-tert-butylcyclohexyl)-2-[(4-phenoxyphenyl)imino]-1,3-oxazolidin-4-yl}benzoyl)-beta-alanine

### Example 7A

The product from Example 4A (0.175 g, 0.23 mmol) was dissolved in THF (1.5 mL) and cooled to 0° C. (ice/water). A solution of tetrabutylammonium fluoride (0.345 μL, 0.345 mmol) in THF was added dropwise and reaction stirred for 30 minutes. The reaction was partitioned between ethyl acetate (15 mL) and saturated ammonium chloride (4 mL). The organic phase was separated, dried (MgSO₄) and purified by flash column chromatography (silica gel, 100:0→50:50 hexanes:ethyl acetate). The purified intermediate (0.054 g, 0.085 mmol) was dissolved in THF (3 mL) and cooled to 0° C. (ice/water). Added solid potassium tert-butoxide (0.023 g, 0.21 mmol) followed by polymer supported 4-toluenesulfonyl chloride (0.07 g, 0.1 mmol) and stirred for 90 minutes. The reaction was quenched with water (1 ml) and stirred for a further 1 hour. The reaction was filtered and solid ammonium chloride (0.02 g, 0.3 mmol) was added and stirred for 30 minutes. The solvent was evaporated under reduced pressure and crude product purified by reverse phase preparative HPLC (20:80→100:0 acetonitrile:0.1% TFA in water) on a YMC ODS Guardpak column to provide the title compound as a white solid (0.023 g, 46%). ¹H NMR (300 MHz, CDCl₃) δ 7.58 (d, J is 8 Hz, 2H), 7.36 (d, J is 8.3 Hz, 2H), 7.23 (d, J is 7.36 Hz, 2H), 7.15 (d, J is 8.9 Hz, 2H), 7.08 (t(br), 1H), 7.01 (t, J is 7.34 Hz, 1H), 6.81 (m, 4H), 4.65 (br 1H), 4.0 (m, 1H), 4.12 (d, J is 10.7 Hz, 1H), 3.9 (t(br), 1H), 3.59 (m, 2H), 2.58 (m, 2H), 1.5 (m, 9H), 0.8 (s, 9H); MS (ESI+) m/z 390 (M−195+H)⁺.

### Example 8

### N-{4-[1 tert-butylcyclohexyl)-2-oxoimidazolidin-4-yl]benzoyl}-beta-alanine

### Example 8A

### 3-{4-[1-(trans-4-tert-Butyl-cyclohexylamino)-2-(tert-butyl-dimethyl-silanyloxy)-ethyl]-benzoylamino}-propionic acid ethyl ester

The crude product from Example 2E (3.85 g, with a trans/cis of approximately 3.5 as determined by ¹HNMR) was purified by flash column chromatography (silica gel, 100:0→50:50) hexanes:ethyl acetate) to isolate pure trans product (0.453 g, 11.5%).

### Example 8B

### 3-{4-F1-[3-(4-Bromo-phenyl)-1-(trans-4-tert-butyl-cyclohexyl)-ureidol-2-(tert-butyl-dimethyl-silanyloxy)-ethyl]-benzoylamino}-propionic acid ethyl ester

The product from Example 8A (0.419 g, 0.79 mmol) was dissolved THF (3 mL) 4-bromophenyl isocyanate (0.155 g, 79 mmol) was added and reaction stirred for 2 hours. The solvent was evaporated under reduced pressure and product purified by flash column chromatography (silica gel, 100:0→70:30 hexanes:ethyl acetate) to provide the title compound as an oil (0.51 g, 0.7 mmol).

### Example 8C

The product from Example 8B (0.39 g, 0.53 mmol) was dissolved in THF (7 mL) and cooled to 0° C. (ice/water). A solution of tetrabutylammonium fluoride (0.8 mL, 0.8 mmol) in THF was added dropwise and reaction stirred for 30 minutes. Then solid potassium tert-butoxide (0.2 g, 1.819 mmol) was added at 0° C. followed by 4-toluenesulfonyl chloride (0.122 g, 0.64 mmol) in THF (1.5 mL) and reaction stirred for 30 minutes. To the reaction was added 2 N sodium hydroxide (1 mL) and stirred for 2 hours. The reaction was quenched with 2 N hydrochloric acid (1.5 ml) and solvent evaporated under reduced pressure. The residue was dissolved in 3:1 acetonitrile:water (4 mL) and purified by reverse phase preparative HPLC (20:80→100:0 acetonitrile:0.1% TFA in water) on a YMC ODS Guardpak column to provide the title compound as a white solid (0.052 g, 17%). ¹H NMR (300 MHz, CDCl₃) δ 7.78 (d, J is 8.14 Hz, 2H), 7.45 (d, J is 8.13 Hz, 2H), 7.42 (s, 4H), 6.83 (t, J is 6.10 Hz, 1H), 4.79 (dd, J is 5.8, 3.73 Hz, 1H), 4.13 (t, J is 9.5 Hz, 1H), 3.37 (m, 2H), 3.70 (m, 1H), 3.51 (dd, J is 5.9, 3.4 Hz, 1H), 2.74 (t, J is 5.8 Hz, 2H), 1.8 (m, 3H), 1.6 (m, 3H), 1.0 (m, 3H), 0.78 (s, 9H); MS (ESI+) m/z 571 (M+H)⁺.

### Example 9

### N-{4-[1-(1,1′-biphenyl-4-yl)-3-(4-tert-butylcyclohexyl)-2-oxoimidazolidin-4-yl]benzoyl}-beta-alanine

### Example 9A

The product from Example 8C (0.041 g, 0.071 mmol), phenylboronic acid (0.013 g, 0.11 mmol), potassium phosphate (0.038 g, 0.18 mmol), tri-O-tolylphosphine (0.002 g, 6.5 micromol), palladium acetate (0.5 mg, 1.4 micromol) was stirred in THF (1.2 mL) and water (0.5 mL). Nitrogen was bubbled through the reaction for 10 minutes and then heated to 65° C. for 30 minutes. Added 1 N HCl (1 mL) and solvent evaporated under reduced pressure. The residue was dissolved in CH₂Cl₂ (2 mL) and purified by flash column chromatography (silica gel, 80:20 CH₂Cl₂;MeOH) to provide the title compound as a white solid (0.024 g, 60%). ¹H NMR (300 MHz, CDCl₃) δ 8.59 (br, 1H), 7.85 (d, J is 8 Hz, 2H), 7.65 (m, 5H), 7.53(d, J is 8.5 Hz, 2H), 7.43(t, J is 7.5 Hz, 2H), 7.3 (m, 2H), 4.95 (m, 1H), 4.27 (t, J is 9 Hz, 1H), 3.5(m, 3H), 2.4 (br, 1H), 1.5 (m, 9H), 0.78 (s, 9H); MS (APCI+) m/z 568 (M+H)⁺.

### Example 10

### N-{4-[(2Z)-2-(1,1′-biphenyl-4-ylimino)-3-(4-tert-butylcyclohexyl)-1,3-oxazolidin-4-yl]benzoyl}-beta-alanine

### Example 10A

The product from Example 2E (0.1 g, 0.18 mmol) was dissolved in THF (1.5 m]L) and 4-biphenyl isocyanate (0.37 g, 0.18 mmol) was added and reaction stirred for 3 hours. The reaction was cooled to 0° C. (ice/water) and a solution of tetrabutylammonium fluoride (0.27 mL, 0.27 mmol) in THF was added dropwise and reaction stirred for 30 minutes. At 0° C. added solid potassium tert-butoxide (0.06 g, 0.54 mmol) followed by polymer supported 4-toluenesulfonyl chloride (0.176 g, 0.27 mmol) and stirred for 90 minutes. Added water (1 mL) and sodium hydroxide (0.25 g, 0.6 mmol) and stirred for 3 hours. The reaction was neutralized with 1 N HCl (1.5 mL) and solvent evaporated to dryness. The residue was dissolved in 3:1 acetonitrile:water (4 mL) and purified by reverse phase preparative HPLC (20:80100:0 acetonitrile:0.1% TFA in water) on YMC ODS Guardpak column to provide the title compound as a white solid (0.006 g, 6%). ¹H NMR (300 MHz, CDCl₃) δ 7.88 (d, J is 7.8 Hz, 2H), 7.6 (m, 6H), 7.45 (m, 4H), 7.38 (m, 1H), 6.98 (t, J is 8 Hz, 1H), 5.25 (m, 1H), 5.0 (t, H is 9 Hz, 1H), 4.59 (m, 1H), 4.35 (br, 1H), 3.7 (m, 2H), 2.65 (t, J is 8 Hz, 2H), 1.5 (m, 9H), 0.71 (s, 9H); MS (ESI+) m/z 568 (M+H)⁺.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A compound having formula (I) 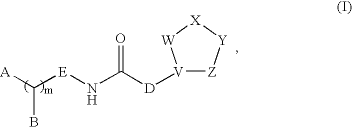
or a pharmaceutically suitable salt, ester or prodrug thereof, wherein
A is selected from the group consisting of CO₂H and tetrazole
B is selected from the group consisting of H, F, OH, alkoxy and —N(RₐR_{b})— wherein Rₐ and R_{b} are each independently selected from the group consisting of hydrogen, alkyl, alkylcarbonyl, alkylsulfonyl alkoxyalkyl, cycloalkyl, cycloalkylcarbonyl, cycloalkylsulfonyl, cycloalkylalkyl, heterocycle, heterocyclealkyl, heterocyclecarbonyl and heterocyclesulfonyl;
D is selected from the group consisting of aryl and heteroaryl;
E is —(CH₂)ₙ—;
m and n are each independently 0, 1, or 2;
V is selected from the group consisting of —C(R)— and —N—, wherein R_{c} is selected from the group consisting of hydrogen, alkyl, alkoxy, alkoxyalkyl, cycloalkyl, cycloalkyloxy, cycloalkylalkyl, heterocycle and heterocyclealkyl;
W is selected from the group consisting of —C(R_{d}Rₑ)—, —(R_{d})N—, —O—, —S—, —S(O)—, and —S(O)₂—;
X is selected from the group consisting of —C(O)—, —C(O)C(R_{f}R_{g})—, —C(R_{f}R_{g})C(O)—, —C(S)—, —C(R_{f}R_{g})—, —C(R_{f}R_{g})C(RᵢRⱼ)—, —C═N(Rⱼ)—, —S(O)— and —S(O)₂—;
Y is selected from the group consisting of —C(RₖRₘ)—, —(Rₖ)N—, —O—, —S—, —S(O)— and —S(O)₂—;
Z is selected from the group consisting of a bond, —C(RₚR_{q})— and —C(RₚR_{q})C(RₛRₜ)—; and
R_{d}, Rₑ, R_{f}, R_{g}, Rᵢ, Rⱼ, Rₖ, Rₘ, Rₚ, R_{q}, Rₛ and Rₜ are each independently selected from the group consisting of hydrogen, alkyl, alkoxy, alkoxyalkyl, aryl, arylalkyl, aryloxy, arylalkoxy, cycloalkyl, cycloalkylalkyl, cycloalkyloxy, cycloalkylalkyl, heterocycle, heterocyclealkyl, heterocycleoxy, and heterocyclealkoxy.

2. The compound according to claim 1 wherein
m is 1;
n is 1;
A is CO₂H;
Bis H; and
D is phenyl.

3. The compound according to claim 1 wherein
m is 1;
n is 1;
A is CO₂H;
B is H;
D is phenyl;
W is (R_{d})N—;
X is —C(O)—;
V is —C(R)—;
Y is —(Rₖ)N—; and
Z is —C(RₚR_{q})—.

4. The compound according to claim 1 wherein
m is 1;
n is 1;
A is CO₂H;
B is H;
D is phenyl;
W is —(R_{d})N—;
X is —C(O)—;
V is —C(R_{c})—;
Y is —(Rₖ)N—;
Z is —C(RₚR_{q})—; and
R_{d} is t-butylphenyl.

5. The compound according to claim 4 wherein the compound is
N-(4-{3-(4-tert-butylphenyl)-2-oxo-1-[4-(trifluoromethoxy)phenyl]imidazolidin-4-yl}benzoyl)-beta-alanine.

6. The compound according to claim 1 wherein
m is 1;
n is 1;
A is CO₂H;
B is H;
D is phenyl;
W is —(R_{d})N—;
X is —C(O)—;
V is —C(R_{c})—;
Y is —(Rₖ)N—;
Z is —C(RₚR_{q})—; and
R_{d} is selected from the group consisting of cis 4-t-butylcyclohexyl and trans 4-t-butylcyclohexyl.

7. The compound according to claim 6 wherein the compound is selected from the group consisting of
N-(4-{3-(4-tert-butylcyclohexyl)-2-oxo-1-[4-(trifluoromethoxy)phenyl]imidazolidin-4-yl}benzoyl)-beta-alanine;
N-{4-[1-(4-bromophenyl)-3-(4-tert-butylcyclohexyl)-2-oxoimidazolidin-4-yl]benzoyl}-beta-alanine;
N-{4-[3-(4-tert-butylcyclohexyl)-2-oxo-1-(4-phenoxyphenyl)imidazolidin-4-yl]benzoyl}-beta-alanine;
N-{4-[1-(4-bromophenyl)-3-(4-tert-butylcyclohexyl)-2-oxoimidazolidin-4-yl]benzoyl}-beta-alanine; and
N-{4-[1-(1,1′-biphenyl-4-yl)-3-(4-tert-butylcyclohexyl)-2-oxoimidazolidin-4-yl]benzoyl}-beta-alanine.

8. The compound according to claim 1 wherein
m is 1;
n is 1;
A is CO₂H;
B is H;
D is phenyl;
W is —(R_{d})N—;
X is —C(O)—;
V is —C(R_{c})—;
Y is —(Rₖ)N—; and
Z is —C(RₚR_{q})C(RₛRₜ)—.

9. The compound according to claim 1 wherein
m is 1;
n is 1;
A is CO₂H;
B is H;
D is phenyl;
W is —(R_{d})N—;
X is —C═N(Rⱼ)—;
V is —C(R_{c})—;
Y is O; and
Z is —C(RₚR_{q})—.

10. The compound according to claim 1 wherein
m is 1;
n is 1;
A is CO₂H;
B is H;
D is phenyl;
W is —(R_{d})N—;
X is —C═N(Rⱼ)—;
V is —C(R_{c})—;
Y is O;
Z is —C(RₚR_{q})—; and
R_{d} is t-butylphenyl.

11. The compound according to claim 1 wherein
m is 1;
n is 1;
A is CO₂H;
B is H;
D is phenyl;
W is —(R_{d})N—;
X is —C═N(Rⱼ)—;
V is —C(R_{c})—;
Y is O;
Z is —C(RₚR_{q})—; and
R_{d} is selected from the group consisting of cis 4-t-butylcyclohexyl and trans 4-t-butylcyclohexyl.

12. The compound according to claim 11 wherein the compound is selected from the group consisting of
N-[4-((2Z)-3-(4-tert-butylcyclohexyl)-2-{[4-(trifluoromethoxy)phenyl]imino}-1,3-oxazolidin-4-yl)benzoyl]-beta-alanine;
N-{4-[(2Z)-2-[(4-bromophenyl)imino]-3-(4-tert-butylcyclohexyl)-1,3-oxazolidin-4-yl]benzoyl}-beta-alanine;
N-(4-{(2Z)-3-(4-tert-butylcyclohexyl)-2-[(4-phenoxyphenyl)imino]-1,3-oxazolidin-4-yl}benzoyl)-beta-alanine; and
N-{4-[(2Z)-2-(1,1′-biphenyl-4-ylimino)-3-(4-tert-butylcyclohexyl)-1,3-oxazolidin-4-yl]benzoyl}-beta-alanine.

13. The compound according to claim 1 wherein
m is 1;
n is 1;
A is CO₂H;
B is H;
D is phenyl;
W is —(R_{d})N—;
X is —C═N(Rⱼ)—;
V is —C(R_{c})—;
Y is —(Rₖ)N—; and
Z is —C(RₚR_{q})—.

14. A pharmaceutical composition comprising a therapeutically effective amount of the compound of claim 1 in combination with a pharmaceutically suitable carrier.

15. A method of selectively antagonizing the glucagon receptor in a mammal comprising administering an effective amount of the compound of claim 1.

16. A method of treating type 2 diabetes in a mammal comprising administering a therapeutically effective amount of the compound of claim 1.

17. A method of treating symptoms related to type 1 or type 2 diabetes in a mammal wherein said symptoms are selected from the group consisting of hyperglycemia, hyperinsulinemia, inadequate glucose clearance, obesity, hyperlipidemia, lipid metabolism disorders and hypertension comprising administering a therapeutically effective amount of the compound of claim 1.

18. A method of treating diabetes or Syndrome X, comprising administration of the compound of formula (I) of claim 1 in combination with an existing anti-diabetic agent selected from the group consisting of insulin, mecasermin, nateglinide, metformin, chlorpropamide, glipizide, glyburide, troglitazone, pioglitazone, rosiglitazone, acarbose, voglibose, miglitol, zopolrestat and repaglinide.

19. A method of treating obesity comprising administrating the compound of formula (I) of claim 1 in combination with an anti-obesity agent selected from the group consisting of orlistat, sibutramine, dexfenfluramine, bromocryptine, phentermine, phendimetrazine and mazindol.
